(19) United States
(12) Patent Application Publication
(10) Pub. No.: US 2012/0020885 A1
(43) Pub. Date: Jan. 26, 2012

(54) MHC-Less cells

(75) Inventors: Roderick A. Hyde, Redmond, WA (US); Muriel Y. Ishikawa, Livermore, CA (US); Edward K.Y. Jung, Bellevue, WA (US); Wayne R. Kindsvogel, Seattle, WA (US); Eric C. Leuthardt, St. Louis, MO (US); Stephen L. Malaska, Redmond, WA (US); Gary L. McKnight, Bothell, WA (US); Elizabeth A. Sweeney, Seattle, WA (US); Lowell L. Wood, JR., Bellevue, WA (US)

(73) Assignee: Searete LLC, a limited liability corporation of the State of Delaware

(21) Appl. No.: 12/806,338

(22) Filed: Aug. 9, 2010

Related U.S. Application Data
(63) Continuation-in-part of application No. 12/804,650, filed on Jul. 26, 2010.
Application No. 12/804,650 is a continuation-in-part of application No. 12/804,649, filed on Jul. 26, 2010.
Application No. 12/804,649 is a continuation-in-part of application No. 12/804,647, filed on Jul. 26, 2010.
Application No. 12/804,647 is a continuation-in-part of application No. 12/804,640, filed on Jul. 26, 2010.
Application No. 12/804,640 is a continuation-in-part of application No. 12/804,648, filed on Jul. 26, 2010.

(51) Int. Cl. A61K 49/00; A61P 35/00; A61P 35/02; A61P 37/06; C12N 5/00; A61K 35/12; A61P 31/00; A61P 9/10; A61P 3/10; A61P 1/12; A61P 17/02; A61P 3/04; C12Q 1/02; A61P 1/02

(52) U.S. Cl. 424/9.1; 435/29; 435/375; 435/325; 424/93.21

(57) ABSTRACT

The present disclosure relates to compositions, methods, systems, computer-implemented methods, and computer program products thereof that relate to biological cells for delivery of at least one therapeutic agent to a biological tissue or subject.

### CROSS-REFERENCE TO RELATED APPLICATIONS

The present application is related to and claims the benefit of the earliest available effective filing date(s) from the following listed application(s) (the “Related applications”) (e.g., claims earliest available priority dates for other than provisional patent applications or claims benefits under 35 USC §119(e) for provisional patent applications, for any and all parent, grandparent, great-grandparent, etc. applications of the Related application(s)). All subject matter of the Related applications and of any and all parent, grandparent, great-grandparent, etc. applications of the Related applications is incorporated herein by reference to the extent such subject matter is not inconsistent herewith.

### RELATED APPLICATIONS

For purposes of the USPTO extra-statutory requirements, the present application constitutes a continuation-in-part of U.S. patent application Ser. No. to be assigned, Docket No. 1004-002-015-000000, entitled MHC-LESS CELLS, naming Roderick A. Hyde, Muriel Y. Ishikawa, Edward K. Y. Jung, Wayne A. Kindsvogel, Eric C. Leuthardt, Stephen L. Malaska, Gary L. McKnight, Elizabeth A. Sweeney and Lowell L. Wood, Jr. as inventors, filed 26 Jul. 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. to be assigned, Docket No. 1004-002-015A-000000, entitled MHC-LESS CELLS, naming Roderick A. Hyde, Muriel Y. Ishikawa, Edward K. Y. Jung, Wayne A. Kindsvogel, Eric C. Leuthardt, Stephen L. Malaska, Gary L. McKnight, Elizabeth A. Sweeney and Lowell L. Wood, Jr. as inventors, filed 26 Jul. 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. to be assigned, Docket No. 1004-002-015B-000000, entitled MHC-LESS CELLS, naming Roderick A. Hyde, Muriel Y. Ishikawa, Edward K. Y. Jung, Wayne A. Kindsvogel, Eric C. Leuthardt, Stephen L. Malaska, Gary L. McKnight, Elizabeth A. Sweeney and Lowell L. Wood, Jr. as inventors, filed 26 Jul. 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. to be assigned, Docket No. 1004-002-015C-000000, entitled MHC-LESS CELLS, naming Roderick A. Hyde, Muriel Y. Ishikawa, Edward K. Y. Jung, Wayne A. Kindsvogel, Eric C. Leuthardt, Stephen L. Malaska, Gary L. McKnight, Elizabeth A. Sweeney and Lowell L. Wood, Jr. as inventors, filed 26 Jul. 2010, which is currently co-pending, or is an application of which a currently co-pending application is entitled to the benefit of the filing date.

For purposes of the USPTO extra-statutory requirements, the present application constitutes a continuation-in-part of U.S. patent application Ser. No. to be assigned, Docket No. 1004-002-015D-000000, entitled MHC-LESS CELLS, naming Roderick A. Hyde, Muriel Y. Ishikawa, Edward K. Y. Jung, Wayne A. Kindsvogel, Eric C. Leuthardt, Stephen L. Malaska, Gary L. McKnight, Elizabeth A. Sweeney and Lowell L. Wood, Jr. as inventors, filed 26 Jul. 2010, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/weekll/patbene.htm. The present Applicant Entity (hereinafter “Applicant”) has provided above a specific reference to the application(s) from which priority is being claimed as recited, by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

### SUMMARY

The present disclosure relates to compositions, methods, systems, and related devices regarding MHC-less cells, or cells with reduced, inactive, or inefficient histocompatibility antigen related genes or gene products. For example, in an embodiment, MHC-less cells have a diminished or eliminated ability to present antigens. In an embodiment, the surface (e.g., chip, liposome, polymer, biological cell, etc.) of the composition is switchable for the presence or absence of at least one of MHC presentation or function.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 illustrates a representation of human MHC genes.

FIG. 2 illustrates a representation of a vector in a cell.

FIG. 3 illustrates a representation of an inducible nucleic acid construct.

FIG. 4 illustrates a partial view of a particular embodiment of a delivery device disclosed herein.

FIG. 5 illustrates a partial view of various embodiments of the device of FIG. 4.

FIG. 6 illustrates a partial view of various embodiments of the device of FIG. 4.

FIG. 7 illustrates a partial view of various embodiments of the device of FIG. 4.

FIG. 8 illustrates a partial view of various embodiments of the device of FIG. 4.

FIG. 9 illustrates a partial view of various embodiments of the device of FIG. 4.

FIG. 10 illustrates a partial view of various embodiments of the device of FIG. 4.

FIG. 11 illustrates a partial view of various embodiments of the device of FIG. 4.

FIG. 12 illustrates a partial view of a system disclosed herein.

FIG. 13 illustrates a partial view of various embodiments of the system of FIG. 12.

FIG. 14 illustrates a partial view of various embodiments of the system of FIG. 12.

FIG. 15 illustrates a partial view of various embodiments of the system of FIG. 12.

FIG. 16 illustrates a partial view of various embodiments of the system of FIG. 12.

FIG. 17 illustrates a partial view of various embodiments of the system of FIG. 12.

FIG. 18 illustrates a partial view of various embodiments of a computer program product disclosed herein.

FIG. 19 illustrates a partial view of various embodiments of the computer program product of FIG. 18.

FIG. 20 illustrates a partial view of various embodiments of a computer-implemented method disclosed herein.

FIG. 21 illustrates a partial view of various embodiments of the computer-implemented method of FIG. 20.

FIG. 22 illustrates a partial view of various embodiments of the computer-implemented method of FIG. 20.

FIG. 23 illustrates a partial view of various embodiments of the computer-implemented method of FIG. 20.

FIG. 24 illustrates a partial view of various embodiments of the computer-implemented method of FIG. 20.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present disclosure relates to compositions, methods, systems, and related devices regarding MHC-less cells. For example, in an embodiment, MHC-less cells have a diminished or eliminated ability to present antigens. In an embodiment, the surface (e.g., chip, liposome, polymer, biological cell, etc.) of the composition is switchable for the presence or absence of at least one of MHC presentation or function. In an embodiment, the composition is configured for suppression of mitosis of a biological cell. In an embodiment, the composition is configured to convey increased resistance to viral infection of at least one biological cell. In an embodiment, one or more self-limiting mechanisms are included in the composition in order to provide a stop-gap measure for containment or control of the composition. In an embodiment, methods of utilizing the compositions disclosed herein include providing the compositions for therapeutic uses. In an embodiment, methods utilizing the composition disclosed herein include administering the composition to at least one biological tissue.

Presentation of antigens by histocompatibility proteins in a subject's immune system plays a vital role in self-defense. In higher vertebrates, a Major Histocompatibility Complex system is used for antigen presentation to other immune system cells. However, such antigen presentation is also a potentially fatal drawback to organ or tissue transplantation, as the subject's own immune system can reject the “dangerous” or “foreign” antigens of the graft. In this regard, cells or other vehicles of the composition that include reduced or eliminated histocompatibility antigen presentation are desirable for tissue or organ transplantation, as well as other therapies. For example, such MHC-less cells are also useful as a vehicle for providing at least one therapeutic agent to a biological tissue or subject.

In an embodiment, the MHC-less cell of the composition includes at least one modified eukaryotic cell. In an embodiment, the MHC-less cell of the composition includes a modified eukaryotic cell including at least one regulator nucleic acid construct including an operon with an inducible promoter and encoding a regulator gene product, wherein at least one of the regulator nucleic acid construct, or the regulator gene product is sufficient to modulate the expression of at least one endogenous modified eukaryotic cell histocompatibility antigen related gene, the modified eukaryotic cell further including at least one therapeutic nucleic acid construct including an operon and encoding at least one therapeutic agent.

In an embodiment, a composition comprises a modified eukaryotic cell including at least one regulator nucleic acid construct including an operon with an inducible promoter and encoding a regulator gene product that is sufficient to modulate the expression of at least one endogenous modified eukaryotic cell histocompatibility antigen related gene, the modified eukaryotic cell further including at least one cell death-initiating nucleic acid construct including an operon with an inducible promoter and encoding at least one gene product that is sufficient to initiate death of the at least one modified eukaryotic cell. In an embodiment, the modified eukaryotic cell includes at least one cell surface receptor capable of modulating at least one immunological activity.

In an embodiment, a composition comprises a modified eukaryotic cell including at least one modification sufficient sufficient to reduce or eliminate expression of at least one endogenous histocompatibility antigen related gene; and at least one detection material. As indicated herein, in an embodiment, the at least one detection material includes at least one of a photo-activatable substance, nanoparticle, radioactive substance, luminescent substance, colorimetric substance, odorous substance, x-ray absorber, ultrasound absorber, or electronic identification device. In an embodiment, the electronic identification device includes at least one radio frequency identification device (RFID). In an embodiment, the at least one detection material includes at least one of a diamagnetic particle, ferromagnetic particle, paramagnetic particle, super paramagnetic particle, particle with altered isotope, or other magnetic particle.

In an embodiment, a composition, comprises a modified eukaryotic cell including at least one regulatory nucleic acid construct including an operon with an inducible promoter and encoding at least one regulatory gene product, wherein at least one of the regulatory nucleic acid construct or the at least one regulatory gene product is sufficient to modulate the expression of at least one endogenous modified eukaryotic cell histocompatibility antigen related gene, the modified eukaryotic cell further including at least one therapeutic nucleic acid construct including an operon and encoding at least one therapeutic gene product. In an embodiment, the therapeutic gene product includes at least one therapeutic agent.

In an embodiment, a composition comprises a modified eukaryotic cell including at least one adenoviral vector construct including an operon with an inducible promoter and encoding an adenoviral gene product that is sufficient to decrease or eliminate the expression of at least one endogenous modified eukaryotic cell histocompatibility antigen related gene, the modified eukaryotic cell further including at least one therapeutic nucleic acid construct including an operon and encoding at least one therapeutic gene product. In an embodiment, the at least one adenoviral vector construct further includes an operon with an inducible promoter and encoding at least one exogenous histocompatibility antigen related gene product.

In an embodiment, a composition comprises a modified eukaryotic cell includes at least two adenoviral vector constructs, at least one adenoviral vector construct including an operon with an inducible promoter and encoding an adenoviral gene product that is sufficient to decrease or eliminate the expression of at least one endogenous modified eukaryotic cell histocompatibility antigen related gene; and at least one different adenoviral vector construct including an operon with an inducible promoter and encoding a therapeutic gene product.

In an embodiment, a composition includes a modified eukaryotic cell including at least one regulatory nucleic acid construct including an operon with an inducible promoter and encoding a regulatory gene product that is sufficient to modulate the expression of at least one endogenous modified eukaryotic cell histocompatibility antigen related gene, the modified eukaryotic cell further including at least one cell death-initiating nucleic acid construct including an operon with an inducible promoter and encoding a gene product that is sufficient to initiate death of the at least one modified eukaryotic cell.

In an embodiment, a composition includes a modified eukaryotic cell including at least one regulatory nucleic acid construct including an operon with an inducible promoter and encoding a regulatory gene product that is sufficient to modulate the expression of at least one endogenous eukaryotic cell histocompatibility antigen related gene, the modified eukaryotic cell further including at least one cell death-initiating component sufficient to initiate death of the at least one modified eukaryotic cell. As described herein, in an embodiment, the cell death-inducing component includes at least one cell death-initiating nucleic acid construct. In an embodiment, the at least one cell death-inducing component includes at least one energy absorbing structure. In an embodiment, the energy absorbing structure includes at least one of an x-ray absorber, metal nanoparticle, or ultrasound absorber. In an embodiment, the at least one cell death-inducing component includes at least one receptor configured to allow entry of at least one of a toxin or pathogen.

In an embodiment, a composition includes a modified eukaryotic cell including at least one regulatory nucleic acid construct including an operon with an inducible promoter and encoding a regulatory gene product that is sufficient to modulate the expression of at least one endogenous eukaryotic cell histocompatibility antigen related gene, and at least one detection material.

In an embodiment, a composition includes a modified eukaryotic cell including at least one regulatory nucleic acid construct including an operon with an inducible promoter and encoding a regulatory gene product that is sufficient to modulate the expression of at least one endogenous eukaryotic cell histocompatibility antigen related gene, and including at least one cell death-inducing component.

In an embodiment, a composition includes a modified eukaryotic cell including at least one modification sufficient to reduce or eliminate expression of at least one endogenous histocompatibility antigen related gene; and the modified eukaryotic cell further including at least one cell death-initiating nucleic acid construct including an operon and an inducible promoter, and encoding at least one gene product sufficient to initiate death in the at least one modified eukaryotic cell.

In an embodiment, a composition includes a modified eukaryotic cell including at least one modification sufficient to reduce or eliminate expression of at least one endogenous histocompatibility antigen related gene, the modified eukaryotic cell including at least one therapeutic nucleic acid construct including an operon and encoding at least one therapeutic agent.

In an embodiment, the modified eukaryotic cell further includes a rescue nucleic acid construct including an operon with an inducible promoter and encoding at least a portion of one or more of an exogenous histocompatibility antigen related gene, or a homologue thereof, or at least a portion of one or more superantigens; and the modified eukaryotic cell further including at least one therapeutic nucleic acid construct including an operon and encoding at least one therapeutic agent. In an embodiment, the at least one modification includes utilizing at least one of site-directed mutagenesis; homologous recombination; non-homologous recombination; ribozyme manipulation; antisense; incorporation of at least one of a peptide nucleic acid, threose nucleic acid or glycol nucleic acid; or chemical mutagenesis. In an embodiment, the at least one modification includes at least one of a gene mutation or gene deletion.

In an embodiment, the at least one modification includes at least one point mutation in the promoter region or in at least one exon of the at least one endogenous histocompatibility antigen related gene. In an embodiment, the reduced expression of the at least one endogenous histocompatibility antigen related gene includes a lack of measurable expression of the at least one endogenous histocompatibility antigen related gene. In an embodiment, the superantigen includes at least one viral, mycoplasma, or bacterial superantigen. In an embodiment, the superantigen includes at least one product of an exotoxin gene. In an embodiment, the superantigen includes at least one product of a Streptococcal pyrogenic exotoxin gene. In an embodiment, the at least one Streptococcal pyrogenic exotoxin gene includes at least one of SpeA, SpeB, SpeC, SpeF, SpeG, SpeH, SSA, or Smez/Smez-2 gene.

In an embodiment, the regulator nucleic acid construct includes at least one of DNA or RNA. In an embodiment, the regulator nucleic acid construct includes double-stranded DNA or double-stranded RNA. In an embodiment, the regulator nucleic acid construct encodes interfering RNA (RNAi). For example, in an embodiment, the regulator nucleic acid construct encodes at least one of microRNA, shRNA, or siRNA. In an embodiment, the regulator nucleic acid construct includes at least a portion of the K5 gene of Kaposi's sarcoma-associated herpesvirus. In an embodiment, the regulator nucleic acid construct encodes an antisense molecule. In an embodiment, the regulator nucleic acid construct encodes at least a portion of a dominant negative mutant form of the at least one endogenous modified eukaryotic cell histocompatibility antigen related gene. In an embodiment, the at least one dominant negative mutant form of the at least one endogenous modified eukaryotic cell histocompatibility antigen includes a mutant β-2m molecule with a defective MHC1 α1 or α2 domain. In an embodiment, the at least one regulatory nucleic acid construct encodes at least one regulatory gene product configured to increase the expression of at least one endogenous or exogenous histocompatibility antigen related gene. In an embodiment, the at least one regulatory nucleic acid construct encodes at least one regulatory gene product configured to decrease or eliminate the expression of at least one histocompatibility antigen related gene.

In an embodiment, an exogenous histocompatibility antigen related gene includes, for example, a histocompatibility antigen related gene from a source outside of the cell in which it is placed, or has been modified from its original, naturally-occurring state. For example, in an embodiment, an exogenous histocompatibility antigen related gene includes a gene from another cell, biological tissue, or subject different than the origin of the cell into which the gene is placed. In an embodiment, an exogenous histocompatibility antigen related gene includes a synthetic gene construct derived ex vivo. In an embodiment, an exogenous histocompatibility antigen related gene includes an endogenous histocompatibility antigen related gene that has been modified (e.g., in vitro, in vivo, ex vivo, in utero, etc.).

In an embodiment, the modified eukaryotic cell includes two or more regulatory nucleic acid constructs, wherein at least one regulatory nucleic acid construct encodes a regulatory gene product configured to increase the expression of at least one histocompatibility antigen related gene, and wherein at least one different regulatory nucleic acid construct encodes a regulatory gene product configured to decrease or eliminate the expression of the at least one histocompatibility antigen related gene.

In an embodiment, at least one of the regulator nucleic acid construct or the therapeutic nucleic acid construct includes at least one regulatory element. In an embodiment, the at least one regulatory element includes at least one of an activator, enhancer, inducer, repressor, or co-repressor.

In an embodiment, at least one of the activator, inducer, repressor, or co-repressor includes at least one of a carbohydrate or antibiotic. In an embodiment, at least one of the activator, inducer, repressor, or co-repressor includes at least one of arabinose, lactose, maltose, sucrose, glucose, xylose, galactose, rhamnose, fructose, melibiose, starch, inunlin, lipopolysaccharide, arsenic, cadmium, chromium, temperature, light, antibiotic, oxygen level, xylan, nisin, L-arabinose, allolactose, D-glucose, D-xylose, D-galactose, ampicillin, tetracycline, penicillin, pristinamycin, retinoic acid, or interferon.

In an embodiment, a composition comprises a modified eukaryotic cell including at least one regulatory nucleic acid construct including an operon with an inducible promoter and encoding a regulatory gene product that is sufficient to decrease or eliminate expression of at least one endogenous Major Histocompatibility Class I gene, the inducible promoter configured to be inducible by at least one orally administered inducer. Various examples of inducers are described herein above.

In an embodiment, at least one of the nucleic acid constructs utilized with a composition includes at least one vector. In an embodiment, the vector includes at least one of a plasmid, cosmid, artificial chromosome, or viral vector. In an embodiment, the viral vector includes at least one of a retroviral vector, lentiviral vector, adeno-associated viral vector, or adenoviral vector. In an embodiment, the vector further includes a synthetic exon promoter trap. In an embodiment, the regulator gene product is sufficient to modulate the expression of at least one of an endogenous or exogenous histocompatibility antigen related gene. In certain aspects, for example, modulating the expression of the at least one endogenous modified eukaryotic cell histocompatibility antigen related gene includes increasing or decreasing the expression of the gene.

In an embodiment, for example, modulating the expression of the at least one endogenous modified eukaryotic cell histocompatibility antigen related gene includes modulating at least one of transcription, translation, secondary modification, peptide bonding, gene product processing, transport of at least one gene product to a cell surface, display of at least one gene product on a cell surface, embedding at least one gene product in a cell surface, or assembly of the histocompatibility antigen related gene product complex at a cell surface. In an embodiment, the regulator gene product is sufficient to inhibit processing or transport of peptide loading of MHC. In an embodiment, the regulator gene product is sufficient to modulate processing or transport of peptide display in MHC. In an embodiment, the regulator gene product is sufficient to inhibit processing, transport, assembly, loading of epitope display in MHC, or anchoring in cell membrane of at least one MHC component.

In an embodiment, the regulatory gene product is sufficient to induce one or more of dislocation of the MHC class I heavy chain into the cell cytosol, inhibition of peptide translocation by TAP, switching of at least one immunoproteasome subunits, inhibition by binding or degradation by at least one immunoproteasome subunit, inhibition of MHC class I association with TAP, inhibition of MHC class I molecule trafficking, inhibition of endoplasmic reticulum export of MHC class I molecules, inhibition by diversion of MHC class I molecules to the cell lysosome, sequestering MHC class I molecules in the trans-Golgi network, inhibition of processing or transport of MHC I loaded with peptide, or sorting MHC class I molecules into the late endocytic pathway for degradation. In an embodiment, the regulator gene product includes a nucleic acid construct that encodes at least a portion of one or more of an artificial zinc finger protein, US2, US11, US6, ICP47, US3, US10, E19, U21, K3, K5, or Nef proteins.

As published, several proteins are utilized by a biological cell in MHC class I antigen presentation. (See, for example, Hewitt, Immunol. vol. 110: 163-169 (2003), which is incorporated herein by reference.) For example, proteins are proteolytically processed in the cytosol by the proteasome. Id. Peptides produced by the proteasome are translocated into the ER lumen by the transporter associated with antigen processing (TAP) protein. Id. TAP also acts as a scaffold for the final stages of MHC class I assembly. Id. In addition, ER resident protein chaperones facilitate the folding of nascent MHC class I molecules and the MHC class I molecule (i.e., heavy chain and β₂M) binds to TAP in a complex with the chaperones calreticulum and ERP57. Id. Tapasin is required in this interaction which acts as a bridging molecule between the MHC class I/chaperone complex and TAP. Tapasin is also required to facilitate binding of high affinity peptides to the MHC class I molecule. Id. Following peptide loading, MHC class I molecules dissociate from TAP and cluster at export sited on the ER membrane where they are selectively recruited for transport to the Golgi apparatus for trafficking through to the plasma membrane. Id.

In an embodiment, a self-limiting mechanism is included in the cell or other vehicle with reduced or eliminated histocompatibility antigen related gene expression. For example, in an embodiment, the cell further comprises at least one cell death-initiating nucleic acid construct including an operon with an inducible promoter and encoding a gene product that is sufficient to initiate death of the at least one modified eukaryotic cell. In an embodiment, the at least one cell death-initiating nucleic acid construct further includes at least one regulatory element. In an embodiment, the at least one regulatory element includes at least one of an activator, enhancer, inducer, repressor, or co-repressor. In an embodiment, the at least one cell death-initiating nucleic acid construct encodes at least one of programmed cell death 1 gene (PDCD1), programmed cell death 2 gene (PDCD2), programmed cell death 3 gene (PDCD3), programmed cell death 4 gene (PDCD4), programmed cell death 5 gene (PDCD5), programmed cell death 6 gene (PDCD6), programmed cell death 7 gene (PDCD7), programmed cell death 8 gene (PDCD8), programmed cell death 9 gene (PDCD9), programmed cell death 10 gene (PDCD10), programmed cell death 11 gene (PDCD11), programmed cell death 12 gene (PDCD12), caspase gene, rel gene, nuclease gene, methylase gene, Bcl-2-associated X protein (Bax), Bcl-2-associated death promoter (BAD), Bcl-2-homologous antagonist/killer (Bak), Bcl-2-related ovarian killer protein (Bok), Fas ligand, Fas receptor, DNA gyrase gene, or a foreign histocompatibility antigen related gene. In an embodiment, the foreign histocompatibility antigen related gene includes a histocompatibility antigen related gene of a different class or subclass, a histocompatibility antigen related gene of a different serotype, or a histocompatibility antigen related gene of a different species than that of the modified eukaryotic cell.

In an embodiment, the modified eukaryotic cell includes at least one of an autologous modified eukaryotic cell, homologous modified eukaryotic cell, allogeneic cell, syngeneic modified eukaryotic cell, or xenogeneic modified eukaryotic cell. Thus, in an embodiment, the MHC-less cell is obtained from a donor subject, and is provided to a recipient. In some cases, the donor and recipient are the same subject, or same species of subject. In some cases, the donor and recipient are different subjects, or different species of subject. In some cases, the MHC-less cell is synthesized from starting materials and is not a modified cell obtained from a subject. In some cases, the MHC-less cell is cultured in vitro from a cell line or from cell or tissue culture.

In an embodiment, a composition comprises a modified eukaryotic cell including at least one regulatory nucleic acid construct including an operon with an inducible promoter and encoding a regulatory gene product that is sufficient to modulate the surface expression of at least one endogenous modified eukaryotic cell histocompatibility antigen related gene, the modified eukaryotic cell further including at least one telomere nucleic acid construct including an operon with an inducible promoter and encoding at least a portion of a telomeric gene product sufficient to inhibit at least one of telomerase enzyme, or Regulator of Telomere Length (Rtel).

In an embodiment, the modified eukaryotic cell includes at least one somatic cell. In an embodiment, the modified eukaryotic cell includes at least one of a blood cell, muscle cell, nerve cell, fibroblast, adipose cell, stem cell, pluripotent cell, epithelial cell, skin cell, liver cell, spleen cell, oocyte, Sertoli cell, neoplastic cell, hematopoietic stem cell, lymphocyte, thymocyte, neuronal stem cell, sperm cell, retinal cell, pancreatic cell, osteoclast, osteoblast, myocyte, embryonic stem cell, keratinocyte, mucosal cell, mesenchymal stem cell, T cell, B cell, memory T cell, memory B cell, antigen presenting cell, lymphocyte, thymocyte, or other cell. The origin of the modified eukaryotic cell includes, but is not limited to, at least one of in situ, in vitro, in vivo, in utero, in planta, in silico, or ex vivo.

In an embodiment, the origin of the modified eukaryotic cell includes at least one of a mammal, reptile, bird, fish, or amphibian. In an embodiment, the mammal includes at least one of a livestock, pet, zoo animal, undomesticated herd animal, wild animal, aquatic plant or animal, or product animal. In an embodiment, the mammal includes a human. In an embodiment, the origin of the modified eukaryotic cell includes at least one fungus or plant.

In certain instances, it is desirable for the MHC-less cell to be in a state of cell cycle arrest. In an embodiment, it is desirable for the MHC-less cell to be mitotically suppressed. For example, in a certain instance, an MHC-less cell is generated for use with organ transplantation, or stem cell transplantation. If it is necessary for a temporal event or lag to occur prior to introducing the MHC-less cell into the recipient subject or biological tissue, then the MHC-less cell is arrested in its cell cycle or is deliberately kept in a quiescent state until it is desired that the MHC-less cell become functional (e.g. assist in organ transplantation, deliver a therapeutic agent, etc.). Thus, in an embodiment, the composition further comprises at least one cell cycle nucleic acid construct including an operon and encoding at least one cell cycle signal. In an embodiment, the at least one cell cycle signal includes at least one cyclin dependent kinase inhibitor. In an embodiment, the cyclin dependent kinase inhibitor includes at least one member of a cip/kip family or Inhibitor of Kinase 4/Alternative Reading Frame (INK4a/ARF) family. In an embodiment, the cyclin dependent kinase inhibitor includes at least one of p16, p21, p27, p57, or p14ARF.

In an embodiment, it is desirable that the MHC-less cells include an expiration or exhaustion time point. For example, in an embodiment the modified eukaryotic cell further includes at least one telomere nucleic acid construct including an operon with an inducible promoter and encoding at least a portion of a telomeric gene product sufficient to inhibit at least one of telomerase enzyme, or Regulator of Telomere Length (Rtel) gene. In an embodiment, the telomere nucleic acid construct includes at least one of double-stranded DNA or double-stranded RNA. In an embodiment, the telomere nucleic acid construct includes at least one of interfering RNA (RNAi).

In an embodiment, the MHC-less cell exhibits increased resistance to infection by at least one virus. For example, in an embodiment, the MHC-less cell exhibits increased resistance to at least one virus, including at least one lentivirus. In an embodiment, the MHC-less cell exhibits increased resistance to at least one virus, including a retrovirus. In an embodiment, the MHC-less cell exhibits increased resistance to at least one of a human acquired immunodeficiency virus (HIV), simian immunodeficiency virus (SIV), feline immunodeficiency virus (FIV), or feline leukemia virus (FeLV).

In an embodiment, the MHC-less cell is configured to deliver at least one therapeutic agent to a subject or biological tissue. In an embodiment, the at least one therapeutic agent includes at least a portion of one of an organic or inorganic small molecule, proteinoid, nucleic acid, serum protein, plasma protein, monosaccharide, disaccharide, polysaccharide, heavy metal, electrolyte, peptide, polypeptide, protein, glycopeptide, glycolipid, lipoprotein, lipopolysaccharide, sphingolipid, glycosphingolipid, glycoprotein, peptidoglycan, lipid, carbohydrate, metalloprotein, proteoglycan, vitamin, mineral, amino acid, polymer, copolymer, monomer, prepolymer, cell receptor, adhesion molecule, cytokine, chemokine, immunoglobulin, antibody, antigen, extracellular matrix constituent, cell ligand, oligonucleotide, element, hormone, transcription factor, or contrast agent. In an embodiment, the polymer or co-polymer includes at least one of polyester, polylactic acid, polyglycolic acid, cellulose, nitrocellulose, urea, urethane, or other polymer. In an embodiment, the at least one therapeutic agent includes at least one of calcium, carbon, nitrogen, sulfur, nitrate, nitrite, copper, magnesium, selenium, boron, sodium, aluminum, phosphorus, potassium, titanium, chromium, manganese, iron, nickel, zinc, silver, barium, lead, vanadium, tin, strontium, or molybdenum. In an embodiment, the at least one therapeutic agent includes at least one of insulin, calcitonin, lutenizing hormone, parathyroid hormone, somatostatin, thyroid stimulating hormone, vasoactive intestinal polypeptide, tumor necrosis factor, endostatin, angiostatin, anti-angiogenic antithrombin II, fibronectin, prolactin, thrombospondin I, laminin, procollagen, collagen, integrin, steroid, corticosteroid, virus antigen, microorganism antigen, receptor, soluble antigen, cell wall, blood plasma, carbohydrate, adhesion molecule, neurotransmitter, or lipase.

In an embodiment, the virus antigen includes at least one antigen from one or more of a double-stranded DNA virus, single-stranded DNA virus, double-stranded RNA virus, (+) single-strand RNA virus, (−) single-strand RNA virus, single-strand RNA-Reverse Transcriptase virus, or double-stranded DNA-Reverse Transcriptase virus.

In an embodiment, the at least one therapeutic agent includes at least one vaccine. In an embodiment, the at least one vaccine includes at least one of an antigenic peptide, antigenic protein, antigenic proteoglycan, antigenic lipid, antigenic glycolipid, antigenic glycoprotein, or antigenic carbohydrate. In an embodiment, the at least one vaccine includes at least one of an envelope protein, capsid protein, surface protein, toxin, polysaccharide, oligosaccharide, phospholipid, mucin, or enzyme needed to make at least one thereof. In an embodiment, the composition further includes at least one adjuvant.

In an embodiment, the at least one therapeutic agent includes at least one cytokine. In an embodiment, the at least one cytokine includes at one of Interleukin-1, Interleukin-2, Interleukin-3, Interleukin-4, Interleukin-5, Interleukin-6, Interleukin-7, Interleukin-8, Interleukin-9, Interleukin-10, Interleukin-11, Interleukin-12, Interleukin-13, Interleukin-14, Interleukin-15, Interleukin-16, Interleukin-17, Interleukin-18, Interleukin-19, Interleukin-20, Interleukin-21, Interleukin-22, Interleukin-23, Interleukin-24, Interleukin-25, Interleukin-26, Interleukin-27, Interleukin-28A and B, Interleukin-29, Interleukin-30, Interleukin-31, Interleukin-32, Interleukin-33, Interleukin-34, Interleukin-35, Interferon-γ, Interferon-α, Interferon-β, Transforming Growth factor, Granulocyte Macrophage-Colony Stimulating Factor, Macrophage-Colony Stimulating Factor, Scarecrow, Erythropoietin, Granulocyte-Colony Stimulating Factor, Leukemia Inhibitory Factor, Oncostatin M, Ciliary Neurotrophic Factor, Growth Hormone, Prolactin, Fibroblast Growth factor, Nerve Growth factor, Platelet Derived Growth factor, Epidermal Growth factor, Fas, Fas ligand, CD40, CD27, CD4, CD8, CD2, CD3, BLyS, Tumor Necrosis Factor-α, or Tumor Necrosis Factor-β.

In an embodiment, the at least one therapeutic agent includes at least one chemokine. In an embodiment, the at least one chemokine includes at least one of CXCR1, CXCR2, CXCR3, CXCR4, CXCR5, CCR1, CCR2, CCR3, CCR4, CCR5, CCR6, CCR7, CCR8, CCR9, IL-8, GROα, GROβ, GROγ, ENA-78, LDGF-PBP, GCP-2, PF4, Mig, IP-10, SDF-1a/13, BUNZO, STRC33, I-TAC, BLC, BCA-1, MIP-1α, MIP1-β, MDC, TECK, TARC, RANTES, HCC-1, HCC-4, DC-CK1, MIP-3α, MIP-3β, MCP-1, MCP-2, MCP-3, MCP-4, eotaxin, MPIF-2, 1-309, MIP-5, HCC2, MPIF-1, 6CKine, CTACK, MEC, lymphotactin, fractalkine, CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CCL29, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, CXCL18, CXCL19, CXCL20, CXCL21, CXCL22, XCL1, XCL2, XCL3, XCL4, XCL5, CX3CL1, CX3CL2, or CX3CL3.

In an embodiment, the at least one therapeutic agent includes at least one soluble receptor. In an embodiment, the at least one therapeutic agent includes at least one immunoglobulin-receptor fusion construct. In an embodiment, the at least one therapeutic agent includes at least one prodrug or precursor compound. In an embodiment, the at least one prodrug or precursor compound includes at least one glucuronide prodrug. In an embodiment, the at least one glucuronide prodrug includes at least one glucuronide of epirubicin, 5-fluorouracil, 4-hydroxycyclophosphamide, or 5-fluorocytosine. In an embodiment, the at least one prodrug or precursor compound includes 5-(aziridin-1-yl)-2,4-dinitrobenzamide. In an embodiment, the at least one therapeutic agent includes at least one converting enzyme responsive to the at least one prodrug or precursor compound. In an embodiment, the at least one enzyme includes at least one of f3 glucuronidase or cytosine deaminase. In an embodiment, the at least one enzyme includes nitroreductase. In an embodiment, the therapeutic agent includes at least a portion of an antibody expressed on the surface of the modified eukaryotic cell, or released therefrom.

In an embodiment, the composition is formulated for administration to the at least one biological tissue by at least one route including peroral, topical, transdermal, epidermal, intravenous, intraocular, tracheal, transmucosal, intracavity, subcutaneous, intramuscular, inhalation, fetal, intrauterine, intragastric, placental, intranasal, interdermal, intradermal, enteral, parenteral, surgical, or injection. In an embodiment, the intracavity route includes at least one of oral, vaginal, uterine, rectal, nasal, peritoneal, ventricular, or intestinal. In an embodiment, the composition is formulated for administration to at least one location in the at least one biological tissue and is translocatable to at least one other location in the at least one biological tissue. In an embodiment, the composition includes one or more of a suspension, mixture, solution, sol, clathrate, colloid, emulsion, microemulsion, aerosol, ointment, capsule, micro-encapsule, powder, tablet, suppository, cream, device, paste, resin, liniment, lotion, ampule, elixir, spray, syrup, foam, pessary, tincture, detection material, polymer, biopolymer, buffer, adjuvant, diluent, lubricant, disintegration agent, suspending agent, solvent, light-emitting agent, colorimetric agent, glidant, anti-adherent, anti-static agent, surfactant, plasticizer, emulsifying agent, flavor, gum, sweetener, coating, binder, filler, compression aid, encapsulation aid, preservative, granulation agent, spheronization agent, stabilizer, adhesive, pigment, sorbent, nanoparticle, microparticle, or gel. In an embodiment, the composition includes a lyophilized formulation.

In an embodiment, the composition further includes at least one detection material associated with the modified eukaryotic cell. In an embodiment, the at least one detection material includes at least one reporter gene, taggant, contrast agent, sensor, or electronic identification device. In an embodiment, the at least one sensor includes at least one biosensor. In an embodiment, the at least one electronic identification device includes at least one radio frequency identification device. In an embodiment, the reporter gene includes at least one of luciferase, green fluorescent protein, β-galactosidase, or chloramphenicol acetyltransferase. In an embodiment, the at least one detection material includes at least one of a radioactive, luminescent, colorimetric or odorous substance. In an embodiment, the detection material includes at least one of a diamagnetic particle, ferromagnetic particle, paramagnetic particle, super paramagnetic particle, particle with altered isotope, or other magnetic particle.

### Major Histocompatibility Complex

Various higher level organisms utilize an endogenous histocompatibility antigen related gene network for immune defense. Such histocompatibility antigen related gene networks include the Major Histocompatibility Complex (MHC) present in humans and some other animals. The MHC includes various central members located in proximity to specific chromosomal loci, as well as other members functionally related thereto.

For example, in an embodiment, the endogenous histocompatibility antigen related gene includes at least one of a Major Histocompatibility Class I gene, Major Histocompatibility Class II gene, or Major Histocompatibility Class III gene. In an embodiment, the at least one endogenous histocompatibility antigen related gene product includes at least one of β-2 microglobulin, Transporter Associated with Antigen Processing (TAP), MHC class I chain-like gene A (MICA), MHC class I α-1 domain, MHC class I α-2 domain, MHC class I α-3 domain, tapasin, calreticulum, ERP57, HLA-A, HLA-B, HLA-C, or calnexin. In an embodiment, the at least one major histocompatibility gene includes at least one of human leukocyte antigen (HLA), H-Y, H-2, dog leukocyte antigen (DLA), bovine leukocyte antigen (BOLA), equine leukocyte antigen (ELA), swine leukocyte antigen (SLA), Rhesus monkey leukocyte antigen (RhL-A), and chimpanzee leukocyte antigen (ChL-A). In an embodiment, the at least one Major Histocompatibility Class II gene includes at least one of an α domain, or a β domain. In an embodiment, the at least one a chain includes at least one of α-1 domain or α-2 domain. In an embodiment, the at least one β domain includes at least one of β-1 domain or β-2 domain. In an embodiment, the at least one Major Histocompatibility Gene includes at least one of HLA-DPA1, HLA-DPB1, HLA-DRA, HLA-DRB1, HLA-DQA1, HLA-DMA, HLA-DMB, HLA-DOA, HLA-DOB, or HLA-DQB1. In an embodiment, the at least one Major Histocompatibility Gene includes at least one of HLA-A, HLA-B, HLA-C, HLA-D, HLA-E, HLA-F, HLA-G, HLA-H, HLA-J, HLA-K, HLA-L, HLA-P, HLA-T, HLA-U, HLA-V, HLA-W, or HLA-X. As published in the EMBL-EBI Database, there are over three thousand HLA Class I alleles, and over one thousand HLA Class II alleles in humans. (See, the worldwide web at ebi.ac.uk/imgt/hla/stats.html, the subject matter of which is incorporated herein by reference). In an embodiment, one or more of the HLA genes or gene products are utilized.

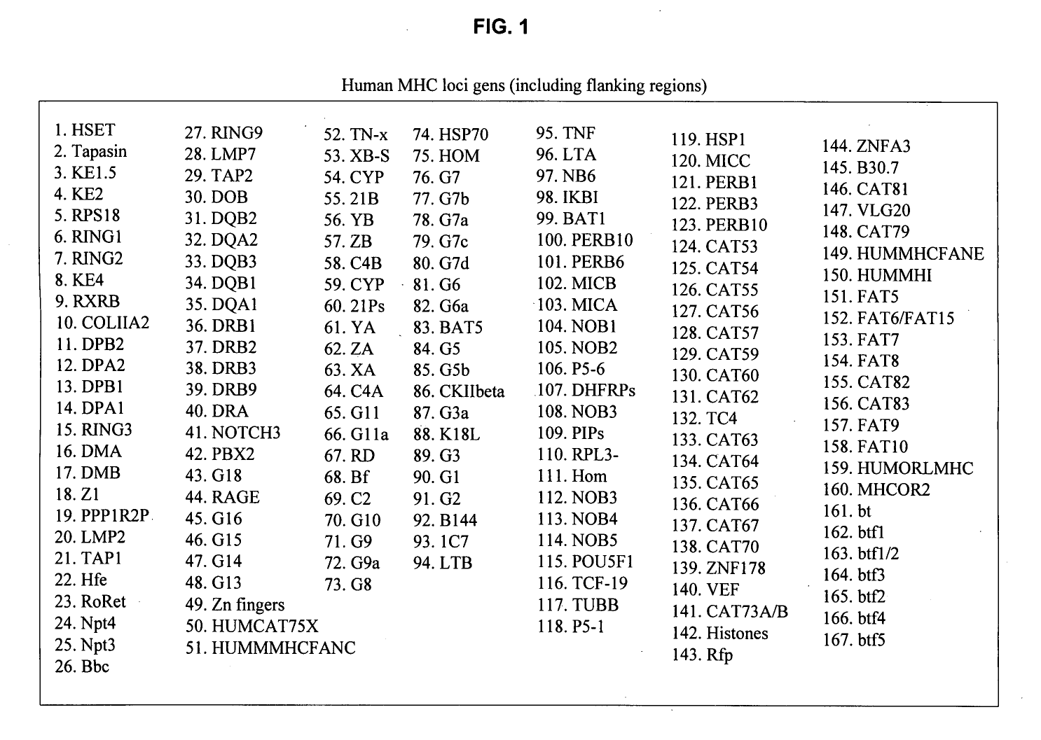

In humans, an extended collection of genes on the short arm of chromosome 6 at 6p21.3 is recognized as the MHC. See FIG. 1, for example. The region is subdivided into three classes, based on certain functional characteristics of the genes for each class. The most centromeric region (class II) contains the HLA-DP, DQ, and DR loci, which are found as pairs, encoding the a and b chains which form the heterodimeric class II protein molecules expressed at the cell surface of antigen presenting cells. These genes are members of the immunoglobulin superfamily (Ig superfamily) of genes. The class I region of the telomeric end contains the classical HLA-A, B, and C family, some of which form heterodimers with the β2 microglobulin chain.

The HLA class I and class II loci are highly polymorphic with many hundreds of allelic variants of several genes. Polymorphism is found particularly common in the α1/2 and β1 domains of HLA class I and II molecules, respectively.

In certain animals, such as chicken, fish, and even invertebrates, multiple pseudogenes lend increased variability to histocompatibility antigen related genes, some of which are functionally or structurally related to their human counterparts.

In an embodiment, a method, therapeutic composition, system, or other embodiment includes modulating NK cells, or means for modulating NK cells. In an embodiment, a composition described herein further includes at least one NK cell inhibitor. For example, it has been published that NK cell-mediated antitumor reactivity (including the release of cytokines, enzymes, and other agents) is based in part, on tumor cells' down-regulation of MHC class I antigen expression, as well as up-regulation of MHC class I chain-related molecule A (MICA) and MICB, which are induced by cellular stress. See, for example, Doubrovina, et al., J. Immunol., vol. 171: 6891-6899 (2003), which is incorporated herein by reference. Thus, in an embodiment, it is desirable to modulate NK cells in order to prevent destruction of the cell of the composition.

In an embodiment, modulating NK cells includes at least one of modulating at least one NK cell-mediated function, including but not limited to cytokine production, production of granzymes, perforins, or proteoglycans, or cell surface receptor recognition of a target molecule (e.g., via an antibody to an NK cell surface receptor, or an antibody to a cell surface receptor on an MHC-less cell disclosed herein). In an embodiment, modulating at least one NK cell-mediated function includes at least partially inhibiting the at least one NK cell-mediated function. In an embodiment, modulating NK cells includes modulating one or more NK cell populations. In an embodiment, modulating NK cells includes reducing or eliminating one or more NK cell populations.

In an embodiment, one or more NK cell populations are depleted using either genetic or epigenetic means. For example, agents that can be used for depletion of NK cells or inhibition of NK cell function include, but are not limited to, an antibody or other agent configured to bind to at least one of P-Selectin Glycoprotein 1 (PSGL-1), human thymocyte globulin, TM-beta1, asialo-GM1, NK1.1, natural cytotoxicity receptor (NCR), leukocyte-associated Ig like receptors (e.g., LAIR-1), killer cell immunoglobulin-like receptor (KIR, e.g., KIR2DL1, KIR2DL2, or KR2DL3).

In an embodiment, the modified eukaryotic cell of the composition includes at least one inhibitory nucleic acid construct encoding at least a portion of an NK cell inhibitor. In an embodiment, the NK cell inhibitor includes at least one of soluble MHC class I chain-related molecule A (MICA), soluble MHC class I chain-related molecule B (MICB), HLA-E, an anti-NK antibody, or other molecule. In an embodiment, the modified eukaryotic cell is at least part of at least one cell mass. In an embodiment, the at least one cell mass includes at least one tumor. In an embodiment, the at least one cell mass includes at least one implant or transplant tissue.

### Methods

Methods of making or administering an MHC-less cell to a biological tissue or subject are disclosed herein. For example, in an embodiment, a method of administering at least one modified cell to at least one biological tissue includes providing a cell composition described herein to at least one biological tissue. In an embodiment, a method of administering at least one therapeutic agent to at least one biological tissue includes providing a cell composition described herein to at least one biological tissue or subject.

In an embodiment, the at least one cell composition is formulated to modulate at least one immune response. In an embodiment, the at least one immune response includes at least one allergic or autoimmune response. In an embodiment, the at least one therapeutic agent is formulated to induce apoptosis in one or more cells of the at least one biological tissue. In an embodiment, the at least one therapeutic agent is formulated to modulate at least one immune response.

In an embodiment, the at least one therapeutic agent is formulated to modulate at least one of viability, proliferation, or metastasis of at least one tumor cell in the at least one biological tissue. In an embodiment, the at least one cell composition is formulated to modulate at least one of viability, proliferation, or metastasis of at least one tumor cell in the at least one biological tissue.

In an embodiment, the at least one biological tissue includes at least one of skin, brain, lung, liver, spleen, bone marrow, thymus, germinal center, heart, myocardium, endocardium, pericardium, lymph node, bone, cartilage, pancreas, kidney, gall bladder, stomach, intestine, testis, uterus, rectum, nervous system, blood, lymph, eye, scalp, nail bed, ear, ovary, oviduct, tongue, tonsil, adenoid, liver, blood vessel, breast, bladder, urethra, ureter, prostate, vas deferens, fallopian tubes, esophagus, oral cavity, nasal cavity, otic cavity, connective tissue, muscle tissue, mucosa-associated lymphoid tissue (MALT), placental tissue, fetal tissue, or adipose tissue. In an embodiment, the at least one biological tissue includes at least one mucosal surface.

In an embodiment, providing the at least one composition includes providing an effective amount of the at least one therapeutic agent in relation to at least one disease, condition, symptom, or disorder. In an embodiment, the at least one composition provides an effective amount of at least one therapeutic agent in relation to at least one of inflammation, infection, immunosuppression, cancer, gastro-intestinal disorder, dental caries, allergic reaction, lactose intolerance, atherosclerosis, diarrhea, fever, anemia, anorexia, autoimmune disease, metabolic defects, diabetes, promoting wound healing, decreasing scar formation, obesity, or malnutrition. In an embodiment, the infection includes at least one of vaginal infection, oral infection, dental infection, urogenital infection, ear infection, eye infection, tonsillitis, ulcer, intestinal blockage or infection, skin infection, nail infection, sinus infection, urinary tract infection, kidney infection, pharyngitis, or laryngitis. In an embodiment, the at least one biological tissue is located in at least one of in situ, in vitro, in vivo, in utero, in planta, in silico, or ex vivo.

In an embodiment, the composition is prepared in vitro prior to providing the composition to the at least one biological tissue. In an embodiment, the at least one modified eukaryotic cell is in physical or chemical communication in vitro with one or more cells of the at least one biological tissue prior to in vivo administration of the at least one modified eukaryotic cell to the at least one biological tissue. In an embodiment, the method further comprises obtaining genetic sequence information from the at least one modified eukaryotic cell. In an embodiment, the genetic sequence information includes information relating to at least one endogenous histocompatibility antigen related gene. In an embodiment, the method further comprises clonally expanding the at least one modified eukaryotic cell prior to administration to the at least one subject. In an embodiment, the at least one biological tissue includes at least one ingestable, implantable, or transplantable biological tissue. In an embodiment, the at least one biological tissue is ingested, transplanted or implanted into at least one subject. In an embodiment, the at least one biological tissue is from at least one eukaryotic or recipient. In an embodiment, the at least one biological tissue includes at least one bodily orifice of a subject.

In an embodiment, the at least one biological tissue includes one or more of cartilage, skin, scalp, hair, nail, nail bed, teeth, eye, ear, ovary, oviduct, tongue, tonsil, adenoid, spleen, lymph node, thymus, liver, bone, pancreas, stomach, duct, valve, smooth muscle, appendix, blood vessel, bone marrow, blood, lymph, heart, lung, brain, breast, kidney, bladder, urethra, ureter, gall bladder, uterus, prostate, testes, vas deferens, fallopian tubes, large intestine, small intestine, esophagus, oral cavity, nasal cavity, otic cavity, connective tissue, muscle tissue, spine, spinal fluid, placental tissue, fetal tissue, or adipose tissue. In an embodiment, the at least one biological tissue includes one or more of a stalk, stem, leaf, root, plant, or tendril. In an embodiment, the at least one biological tissue includes at least one cell mass or wound.

In an embodiment, the at least one biological tissue is at least partially located in at least one subject. In an embodiment, the at least one composition is self-administered by the at least one subject. In an embodiment, the at least one subject includes at least one invertebrate or vertebrate animal. In an embodiment, the at least one subject includes at least one of a reptile, mammal, amphibian, bird, or fish. In an embodiment, the at least one subject includes at least one human. In an embodiment, the at least one subject includes at least one of livestock, pet, zoo animal, undomesticated herd animal, wild animal, aquatic plant or animal, or product animal. In an embodiment, the at least one subject includes at least one of a sheep, goat, frog, dog, cat, rat, mouse, vermin, reptile, monkey, horse, cow, pig, chicken, shellfish, fish, turkey, llama, alpaca, bison, buffalo, ape, primate, ferret, wolf, fox, coyote, deer, rabbit, guinea pig, yak, elephant, tiger, lion, cougar, chinchilla, mink, reindeer, elk, camel, fox, elk, deer, raccoon, donkey, or mule. In an embodiment, the at least one subject includes at least one of a sea anemone, coral, mollusk, fish, whale, dolphin, porpoise, seal, otter, beaver, seabird, gull, pelican, albatross, duck, swan, anthozoan, or goose.

In certain instances, it is desirable to reduce or eliminate the MHC-less cell(s), for example for self-containment. Thus, in an embodiment, the method further includes inducing expression of at least one Fas ligand in the modified eukaryotic cell of the composition. In an embodiment, the method further includes administering at least one anti-Fas antibody.

In an embodiment, the method further includes administering at least one inducer formulated for inducing at least one promoter operably coupled to the at least one cell death-initiating nucleic acid construct. In an embodiment, the method further includes detecting at least one of the presence, amount, concentration, or location of the at least one modified eukaryotic cell subsequent to administration of the composition.

In an embodiment, the method further includes detecting at least one of the presence, amount, concentration, or location of the at least one therapeutic agent subsequent to administration of the composition. In an embodiment, the method further includes selecting for administration an amount or type of composition. In an embodiment, the method further includes selecting for administration an amount or type of at least one of an inducer or repressor of one or more of the regulator nucleic acid construct or therapeutic nucleic acid construct. In an embodiment, the method further includes selecting for administration an amount or type of at least one of an inducer or repressor of the cell death-initiating nucleic acid construct. In an embodiment, the composition is administered by at least one route including one or more of including peroral, topical, transdermal, epidermal, intravenous, intraocular, tracheal, transmucosal, intracavity, subcutaneous, intramuscular, inhalation, fetal, intrauterine, intragastric, placental, intranasal, interdermal, intradermal, enteral, parenteral, surgical, or injection. In an embodiment, administration of the composition includes delivery of the at least one modified eukaryotic cell by way of a device. In an embodiment, the composition is formulated for regulation in vivo. In an embodiment, at least one inducible promoter of the composition is formulated to be induced in vivo.

In an embodiment, a method of increasing immunological tolerance, increasing engraftment, or decreasing rejection of least one biological tissue transplant in a subject includes providing a composition to at least one biological tissue; wherein the composition includes at least one modified eukaryotic cell including at least one regulator nucleic acid construct including an operon with an inducible promoter and encoding a regulator gene product that is sufficient to modulate the expression of at least one endogenous modified eukaryotic cell histocompatibility antigen related gene, the modified eukaryotic cell further including at least one therapeutic nucleic acid construct including an operon and encoding at least one therapeutic agent; and transplanting the at least one biological tissue to a subject.

In an embodiment, a method of treating at least one disease, condition, symptom, or disorder, includes providing an effective amount of a composition to at least one biological tissue of a subject; wherein the composition includes at least one modified eukaryotic cell including at least one regulator nucleic acid construct including an operon with an inducible promoter and encoding a regulator gene product that is sufficient to modulate the expression of at least one endogenous modified eukaryotic cell histocompatibility antigen related gene, the modified eukaryotic cell further including at least one therapeutic nucleic acid construct including an operon and encoding at least one therapeutic agent.

In an embodiment, a method of making a histocompatibility antigen related gene modified eukaryotic cell comprises modifying a modified eukaryotic cell to reduce or eliminate expression of at least one endogenous histocompatibility antigen related gene, the modified eukaryotic cell including at least one rescue nucleic acid construct including an operon with an inducible promoter and encoding at least a portion of one or more of an exogenous histocompatibility antigen related gene product, or a homologue thereof, or at least a portion of one or more superantigens; and the modified eukaryotic cell further including at least one therapeutic nucleic acid construct including an operon and encoding at least one therapeutic agent.

In an embodiment, a method of making a histocompatibility antigen related gene modified eukaryotic cell comprises modifying a modified eukaryotic cell to reduce or eliminate expression of at least one endogenous histocompatibility antigen related gene, the modified eukaryotic cell including at least one rescue nucleic acid construct including an operon with an inducible promoter and encoding at least a portion of one or more of an exogenous histocompatibility related gene product, or a homologue thereof, or at least a portion of one or more superantigens; and the modified eukaryotic cell further including at least one cell death-initiating nucleic acid construct including an operon, and an inducible promoter, and encoding at least one gene product sufficient to initiate death of the at least one modified eukaryotic cell.

As illustrated in FIG. 1, the human MHC and flanking regions include a multiple genes, including histocompatibility antigen related genes, as depicted.

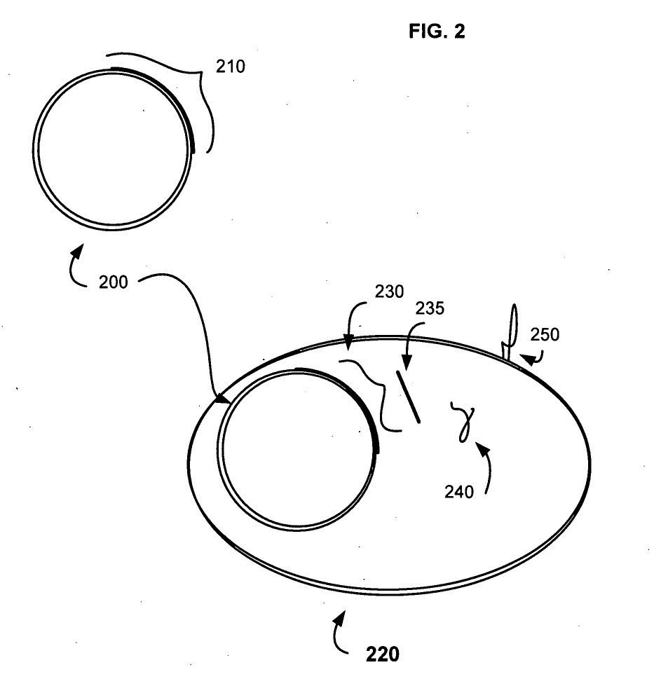

As illustrated in FIG. 2, in an embodiment, a vector 200 including at least one regulatory nucleic acid construct 210 is placed into at least one cell 220 by methods known in the art (e.g., electroporation, transformation, etc.). Once incorporated into the cell 220, the regulatory nucleic acid construct 210 either inhibits proper gene expression of at least one endogenous histocompatibility antigen related gene 235 (e.g., shRNA, iRNA, microRNA, etc.), or is transcribed, resulting in production of at least one transcript 230, which is converted intracellularly into at least one protein 240 (e.g, a dominant negative form of MHC or other histocompatibility antigen related gene). In an embodiment, the protein can remain intracellularly 240, or be expressed on the surface 250 of the cell 220.

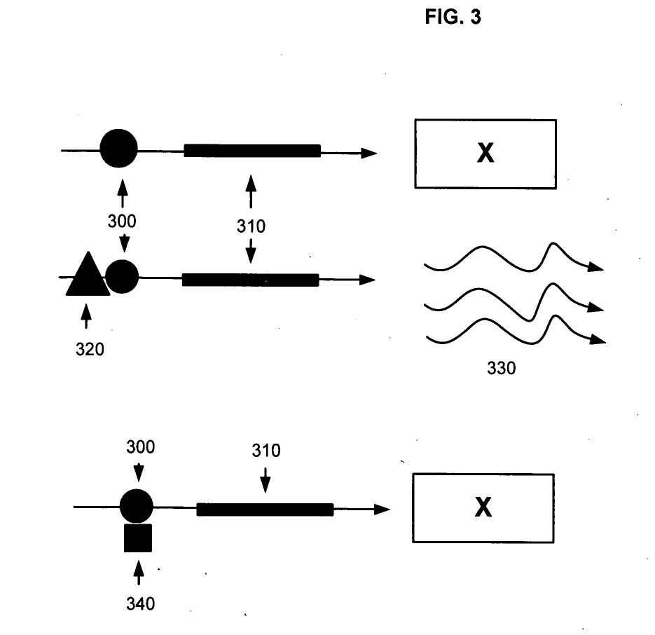

As illustrated in FIG. 3, an example of a nucleic construct, including an inducible promoter 300 is capable of regulating expression of at least one gene 310. In the absence of an inducer 320, the gene 310 is not transcribed (as indicated by the “X”). However, in the presence of the inducer 320, the promoter 300 directs transcription of the gene 310, resulting in production of at least one transcript 330. Likewise, in the presence of a repressor 340, the promoter 300 does not support gene transcription of the gene 310 (as indicated by the “X”).

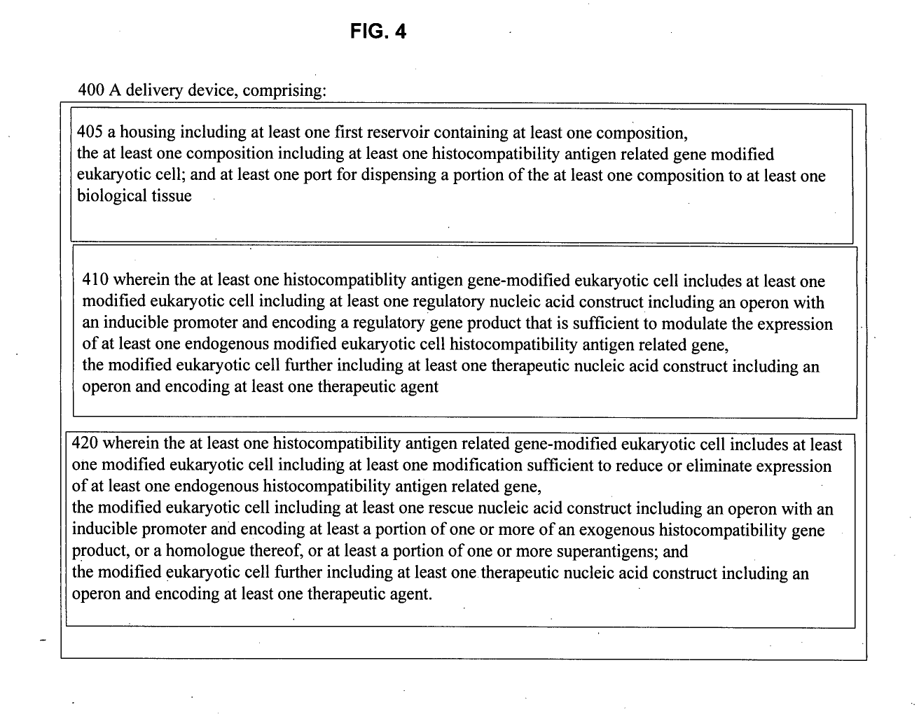

As illustrated in FIG. 4, a delivery device 400, comprises: 405 a housing including at least one first reservoir containing at least one composition, the at least one composition including at least one histocompatibility antigen related gene modified eukaryotic cell; and at least one port for dispensing a portion of the at least one composition to at least one biological tissue. In an embodiment 410, the at least one histocompatiblity antigen gene modified eukaryotic cell includes at least one modified eukaryotic cell including at least one regulatory nucleic acid construct including an operon with an inducible promoter and encoding a regulatory gene product that is sufficient to modulate the expression of at least one endogenous modified eukaryotic cell histocompatibility antigen related gene, the modified eukaryotic cell further including at least one therapeutic nucleic acid construct including an operon and encoding at least one therapeutic agent.

In an embodiment 420, the at least one histocompatibility antigen related gene modified eukaryotic cell includes at least one modified eukaryotic cell including at least one modification sufficient to reduce or eliminate expression of at least one endogenous histocompatibility antigen related gene, the modified eukaryotic cell including at least one rescue nucleic acid construct including an operon with an inducible promoter and encoding at least a portion of one or more of an exogenous histocompatibility gene product, or a homologue thereof, or at least a portion of one or more superantigens; and the modified eukaryotic cell further including at least one therapeutic nucleic acid construct including an operon and encoding at least one therapeutic agent.

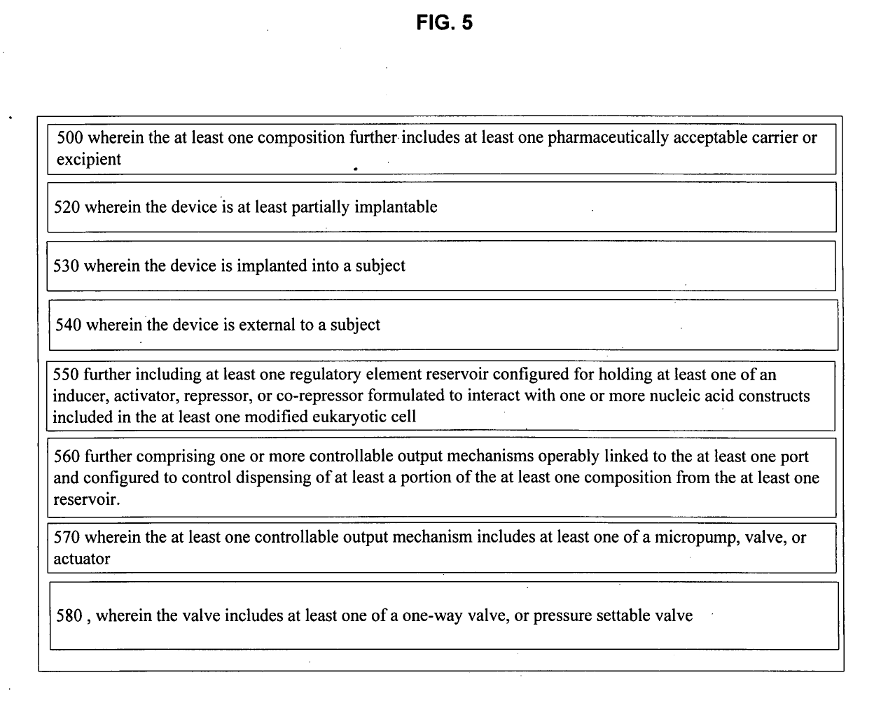

As illustrated in FIG. 5, in an embodiment 500, the at least one composition further includes at least one pharmaceutically acceptable carrier or excipient. In an embodiment 520, the device is at least partially implantable. In an embodiment 530, the device is implanted into a subject. In an embodiment 540, the device is external to a subject. In an embodiment 550, the delivery device further includes at least one regulatory element reservoir configured for holding at least one of an inducer, activator, repressor, or co-repressor formulated to interact with one or more nucleic acid constructs included in the at least one modified eukaryotic cell. In an embodiment 560, the delivery device further includes one or more controllable output mechanisms operably linked to the at least one port and configured to control dispensing of at least a portion of the at least one composition from the at least one reservoir. In an embodiment 570, the at least one controllable output mechanism includes at least one of a micropump, valve, or actuator. In an embodiment 580, the valve includes at least one of a one-way valve, or pressure settable valve.

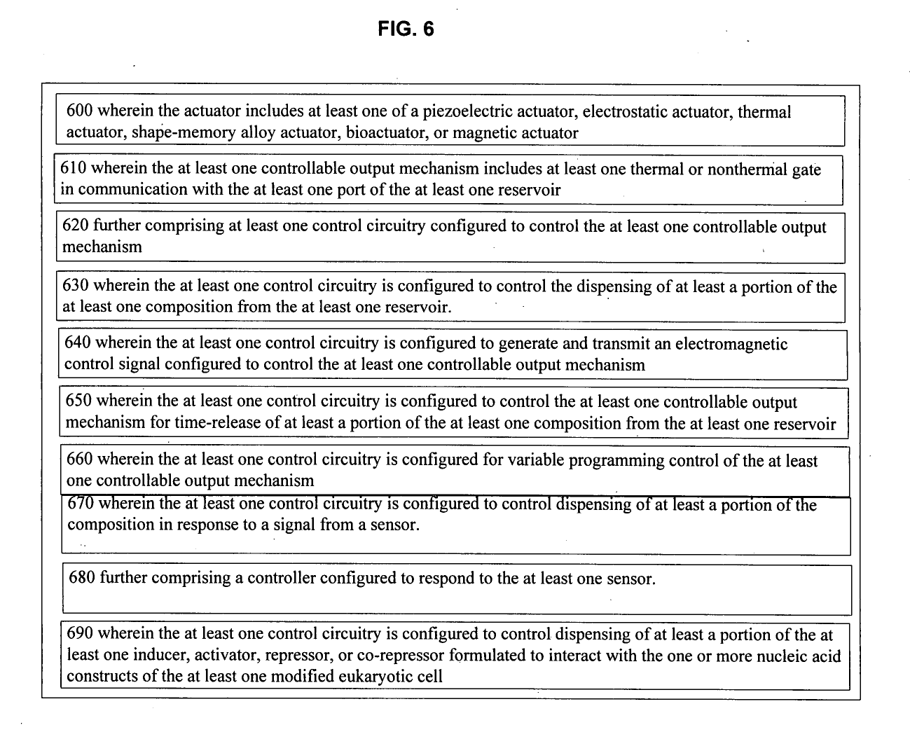

As illustrated in FIG. 6, in an embodiment 600, the actuator includes at least one of a piezoelectric actuator, electrostatic actuator, thermal actuator, shape-memory alloy actuator, bioactuator, or magnetic actuator. In an embodiment 610, the at least one controllable output mechanism includes at least one thermal or nonthermal gate in communication with the at least one port of the at least one reservoir. In an embodiment 620, the delivery device further includes at least one control circuitry configured to control the at least one controllable output mechanism. In an embodiment 630, the at least one control circuitry is configured to control the dispensing of at least a portion of the at least one composition from the at least one reservoir. In an embodiment 640, the at least one control circuitry is configured to generate and transmit an electromagnetic control signal configured to control the at least one controllable output mechanism. In an embodiment 650, the at least one control circuitry is configured to control the at least one controllable output mechanism for time-release of at least a portion of the at least one composition from the at least one reservoir. In an embodiment 660, the at least one control circuitry is configured for variable programming control of the at least one controllable output mechanism. In an embodiment 670, the at least one control circuitry is configured to control dispensing of at least a portion of the composition in response to a signal from a sensor. In an embodiment 680, the delivery device further includes a controller configured to respond to the at least one sensor. In an embodiment 690, the at least one control circuitry is configured to control dispensing of at least a portion of the at least one inducer, activator, repressor, or co-repressor formulated to interact with the one or more nucleic acid constructs of the at least one modified eukaryotic cell.

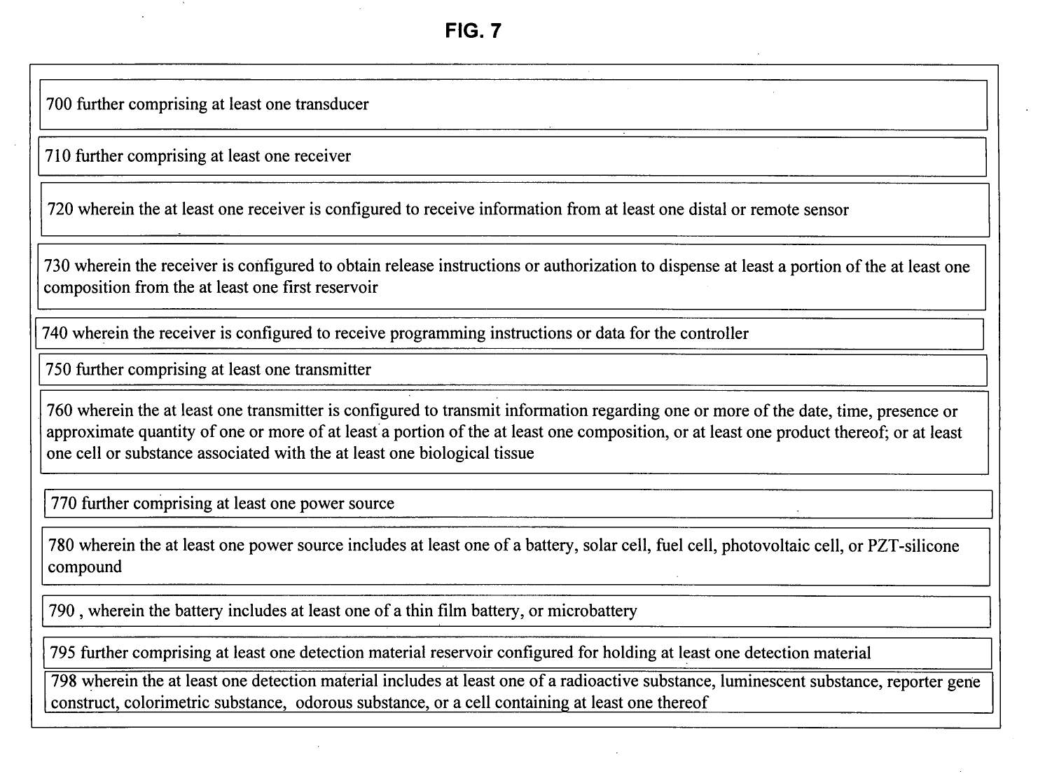

As illustrated in FIG. 7, in an embodiment 700, the delivery device further comprises at least one transducer. In an embodiment 710, the delivery device further comprises at least one receiver. In an embodiment 720, the at least one receiver is configured to receive information from at least one distal or remote sensor. In an embodiment 730, the receiver is configured to obtain release instructions or authorization to dispense at least a portion of the at least one composition from the at least one first reservoir. In an embodiment 740, the receiver is configured to receive programming instructions or data for the controller. In an embodiment 750, the delivery device further comprises at least one transmitter. In an embodiment 760, the at least one transmitter is configured to transmit information regarding one or more of the date, time, presence or approximate quantity of one or more of at least a portion of the at least one composition, or at least one product thereof; or at least one cell or substance associated with the at least one biological tissue. In an embodiment 770, the delivery device further comprises at least one power source. In an embodiment 780, the at least one power source includes at least one of a battery, solar cell, fuel cell, photovoltaic cell, or PZT-silicone compound. In an embodiment 790, the battery includes at least one of a thin film battery, or microbattery. In an embodiment 795, the delivery device further comprises at least one detection material reservoir configured for holding at least one detection material. In an embodiment 798, the at least one detection material includes at least one of a radioactive substance, luminescent substance, reporter gene construct, colorimetric substance, odorous substance, or a cell containing at least one thereof.

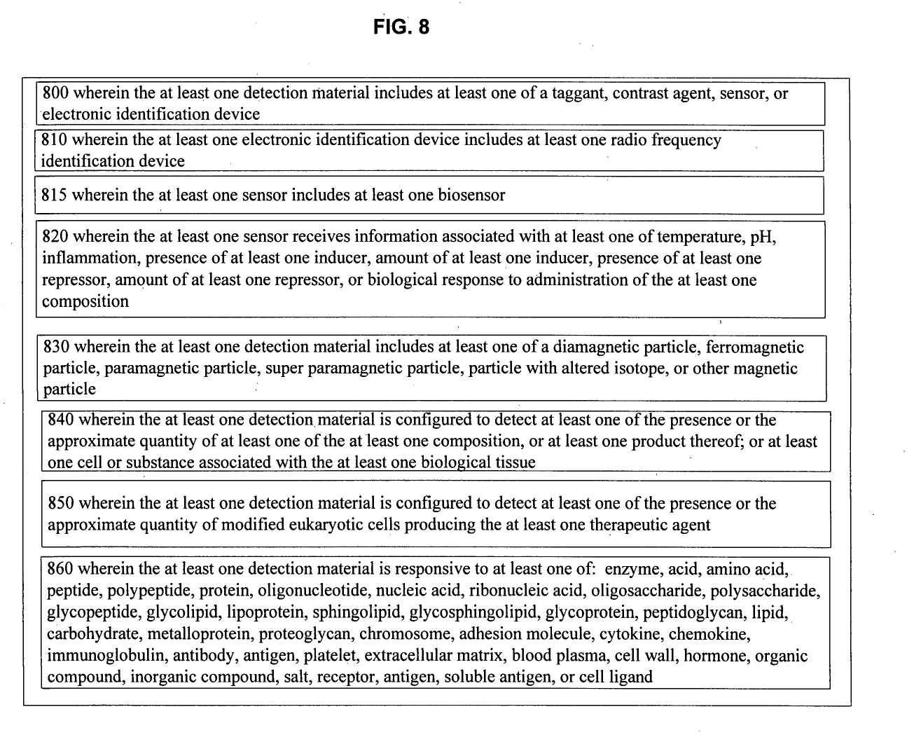

As illustrated in FIG. 8, in an embodiment 800, the at least one detection material includes at least one of a taggant, contrast agent, sensor, or electronic identification device. In an embodiment 810, the at least one electronic identification device includes at least one radio frequency identification device. In an embodiment 815, the at least one sensor includes at least one biosensor. In an embodiment 820, the at least one sensor receives information associated with at least one of temperature, pH, inflammation, presence of at least one inducer, amount of at least one inducer, presence of at least one repressor, amount of at least one repressor, or biological response to administration of the at least one composition. In an embodiment 830, the at least one detection material includes at least one of a diamagnetic particle, ferromagnetic particle, paramagnetic particle, super paramagnetic particle, particle with altered isotope, or other magnetic particle. In an embodiment 840, the at least one detection material is configured to detect at least one of the presence or the approximate quantity of at least one of the at least one composition, or at least one product thereof; or at least one cell or substance associated with the at least one biological tissue. In an embodiment 850, the at least one detection material is configured to detect at least one of the presence or the approximate quantity of modified eukaryotic cells producing the at least one therapeutic agent. In an embodiment 860, the at least one detection material is responsive to at least one of: enzyme, acid, amino acid, peptide, polypeptide, protein, oligonucleotide, nucleic acid, ribonucleic acid, oligosaccharide, polysaccharide, glycopeptide, glycolipid, lipoprotein, sphingolipid, glycosphingolipid, glycoprotein, peptidoglycan, lipid, carbohydrate, metalloprotein, proteoglycan, chromosome, adhesion molecule, cytokine, chemokine, immunoglobulin, antibody, antigen, platelet, extracellular matrix, blood plasma, cell wall, hormone, organic compound, inorganic compound, salt, receptor, antigen, soluble antigen, or cell ligand.

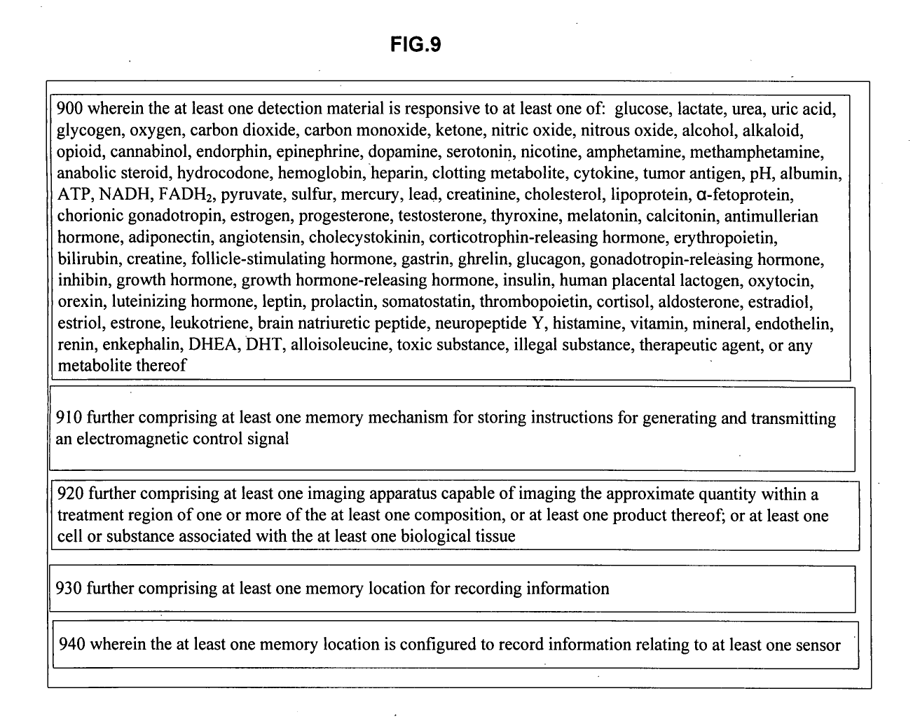

As illustrated in FIG. 9, in an embodiment 900, the at least one detection material is responsive to at least one of: glucose, lactate, urea, uric acid, glycogen, oxygen, carbon dioxide, carbon monoxide, ketone, nitric oxide, nitrous oxide, alcohol, alkaloid, opioid, cannabinol, endorphin, epinephrine, dopamine, serotonin, nicotine, amphetamine, methamphetamine, anabolic steroid, hydrocodone, hemoglobin, heparin, clotting metabolite, cytokine, tumor antigen, pH, albumin, ATP, NADH, FADH₂, pyruvate, sulfur, mercury, lead, creatinine, cholesterol, lipoprotein, α-fetoprotein, chorionic gonadotropin, estrogen, progesterone, testosterone, thyroxine, melatonin, calcitonin, antimullerian hormone, adiponectin, angiotensin, cholecystokinin, corticotrophin-releasing hormone, erythropoietin, bilirubin, creatine, follicle-stimulating hormone, gastrin, ghrelin, glucagon, gonadotropin-releasing hormone, inhibin, growth hormone, growth hormone-releasing hormone, insulin, human placental lactogen, oxytocin, orexin, luteinizing hormone, leptin, prolactin, somatostatin, thrombopoietin, cortisol, aldosterone, estradiol, estriol, estrone, leukotriene, brain natriuretic peptide, neuropeptide Y, histamine, vitamin, mineral, endothelin, renin, enkephalin, DHEA, DHT, alloisoleucine, toxic substance, illegal substance, therapeutic agent, or any metabolite thereof. In an embodiment 910, the delivery device further comprises at least one memory mechanism for storing instructions for generating and transmitting an electromagnetic control signal. In an embodiment 920, the delivery device further comprises at least one imaging apparatus capable of imaging the approximate quantity within a treatment region of one or more of the at least one composition, or at least one product thereof; or at least one cell or substance associated with the at least one biological tissue. In an embodiment 930, the delivery device further comprises at least one memory location for recording information. In an embodiment 940, the at least one memory location is configured to record information relating to at least one sensor.

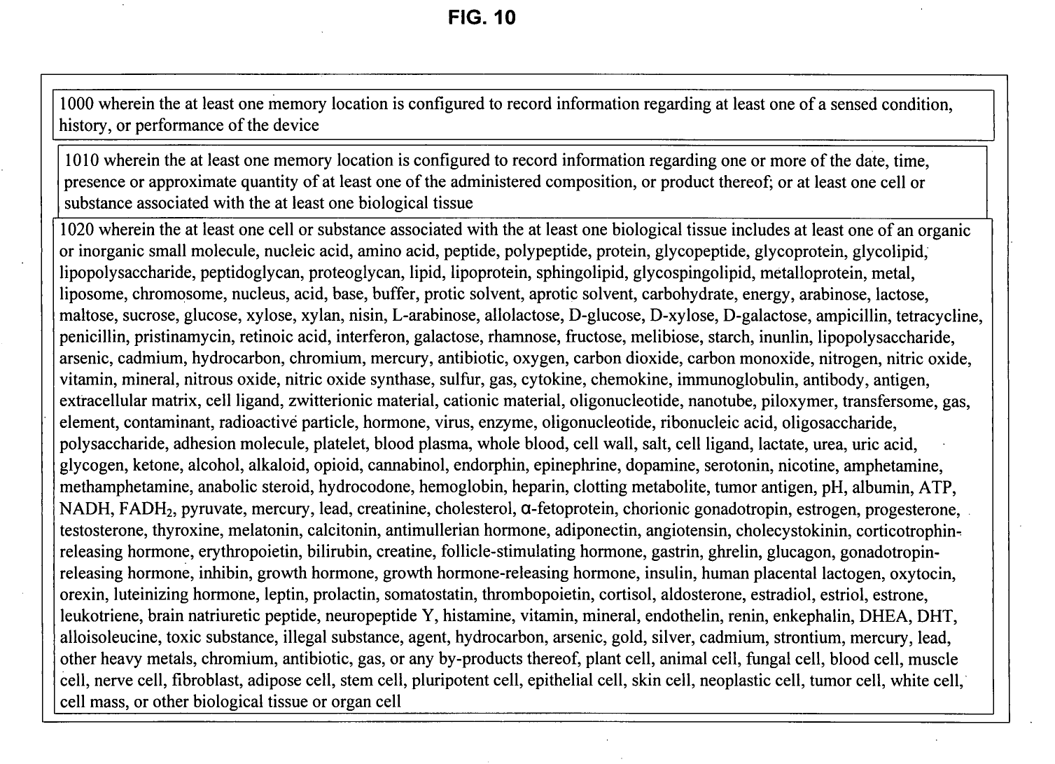

As illustrated in FIG. 10, in an embodiment 1000, the at least one memory location is configured to record information regarding at least one of a sensed condition, history, or performance of the device. In an embodiment 1010, the at least one memory location is configured to record information regarding one or more of the date, time, presence or approximate quantity of at least one of the administered composition, or product thereof; or at least one cell or substance associated with the at least one biological tissue. In an embodiment 1020, the at least one cell or substance associated with the at least one biological tissue includes at least one of an organic or inorganic small molecule, nucleic acid, amino acid, peptide, polypeptide, protein, glycopeptide, glycoprotein, glycolipid, lipopolysaccharide, peptidoglycan, proteoglycan, lipid, lipoprotein, sphingolipid, glycospingolipid, metalloprotein, metal, liposome, chromosome, nucleus, acid, base, buffer, protic solvent, aprotic solvent, carbohydrate, energy, arabinose, lactose, maltose, sucrose, glucose, xylose, xylan, nisin, L-arabinose, allolactose, D-glucose, D-xylose, D-galactose, ampicillin, tetracycline, penicillin, pristinamycin, retinoic acid, interferon, galactose, rhamnose, fructose, melibiose, starch, inunlin, lipopolysaccharide, arsenic, cadmium, hydrocarbon, chromium, mercury, antibiotic, oxygen, carbon dioxide, carbon monoxide, nitrogen, nitric oxide, vitamin, mineral, nitrous oxide, nitric oxide synthase, sulfur, gas, cytokine, chemokine, immunoglobulin, antibody, antigen, extracellular matrix, cell ligand, zwitterionic material, cationic material, oligonucleotide, nanotube, piloxymer, transfersome, gas, element, contaminant, radioactive particle, hormone, virus, enzyme, oligonucleotide, ribonucleic acid, oligosaccharide, polysaccharide, adhesion molecule, platelet, blood plasma, whole blood, cell wall, salt, cell ligand, lactate, urea, uric acid, glycogen, ketone, alcohol, alkaloid, opioid, cannabinol, endorphin, epinephrine, dopamine, serotonin, nicotine, amphetamine, methamphetamine, anabolic steroid, hydrocodone, hemoglobin, heparin, clotting metabolite, tumor antigen, pH, albumin, ATP, NADH, FADH₂, pyruvate, mercury, lead, creatinine, cholesterol, α-fetoprotein, chorionic gonadotropin, estrogen, progesterone, testosterone, thyroxine, melatonin, calcitonin, antimullerian hormone, adiponectin, angiotensin, cholecystokinin, corticotrophin-releasing hormone, erythropoietin, bilirubin, creatine, follicle-stimulating hormone, gastrin, ghrelin, glucagon, gonadotropin-releasing hormone, inhibin, growth hormone, growth hormone-releasing hormone, insulin, human placental lactogen, oxytocin, orexin, luteinizing hormone, leptin, prolactin, somatostatin, thrombopoietin, cortisol, aldosterone, estradiol, estriol, estrone, leukotriene, brain natriuretic peptide, neuropeptide Y, histamine, vitamin, mineral, endothelin, renin, enkephalin, DHEA, DHT, alloisoleucine, toxic substance, illegal substance, agent, hydrocarbon, arsenic, gold, silver, cadmium, strontium, mercury, lead, other heavy metals, chromium, antibiotic, gas, or any by-products thereof, plant cell, animal cell, fungal cell, blood cell, muscle cell, nerve cell, fibroblast, adipose cell, stem cell, pluripotent cell, epithelial cell, skin cell, neoplastic cell, tumor cell, white cell, cell mass, or other biological tissue or organ cell.

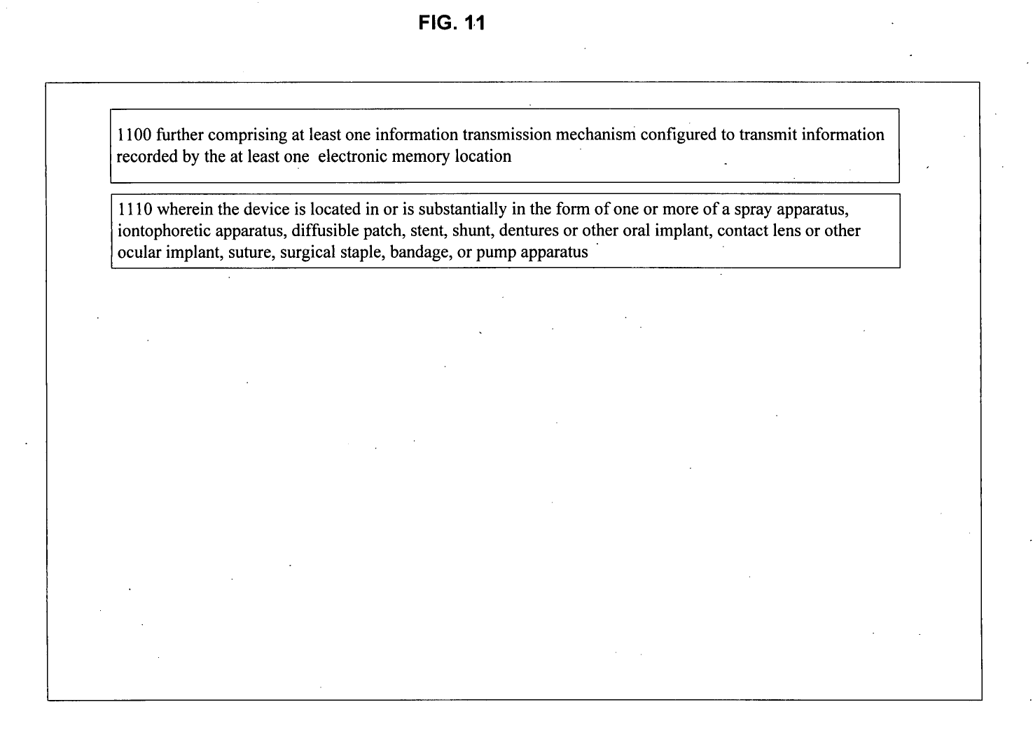

As illustrated in FIG. 11, in an embodiment 1100, the delivery device further comprises at least one information transmission mechanism configured to transmit information recorded by the at least one electronic memory location. In an embodiment 1110, the device is located in or is substantially in the form of one or more of a spray apparatus, iontophoretic apparatus, diffusible patch, stent, shunt, dentures or other oral implant, contact lens or other ocular implant, suture, surgical staple, bandage, or pump apparatus.

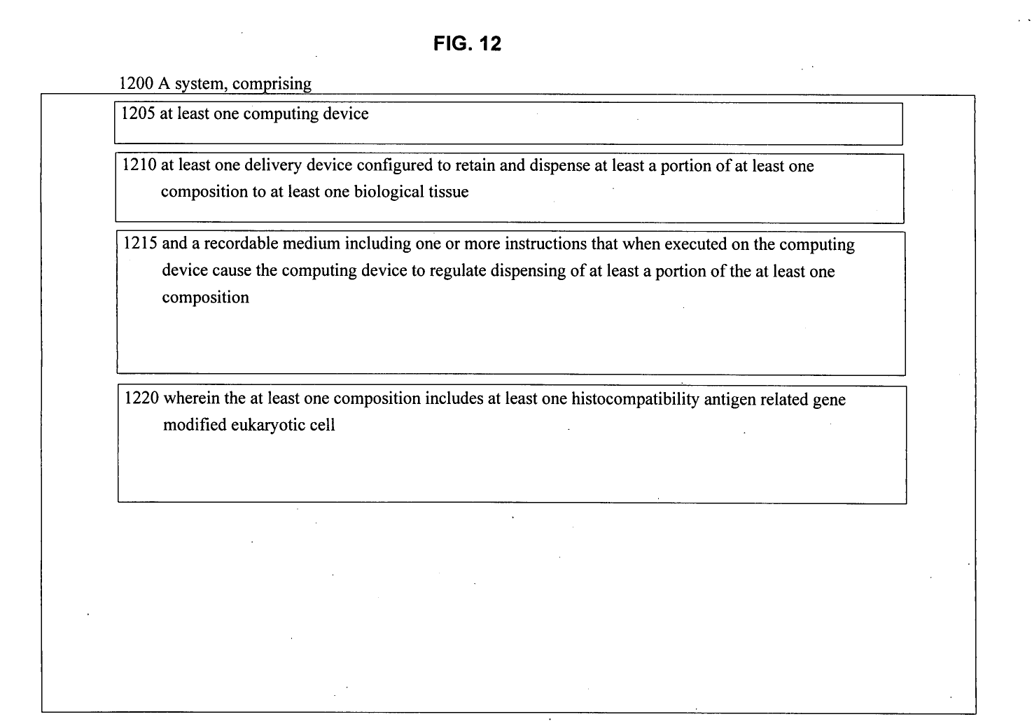

As illustrated in FIG. 12, a system 1200, comprises: 1205 at least one computing device; 1210 at least one delivery device configured to retain and dispense at least a portion of at least one composition to at least one biological tissue; and 1215 a recordable medium including one or more instructions that when executed on the computing device cause the computing device to regulate dispensing of at least a portion of the at least one composition. In an embodiment 1220, the at least one composition includes at least one histcompatibility antigen related gene modified eukaryotic cell.

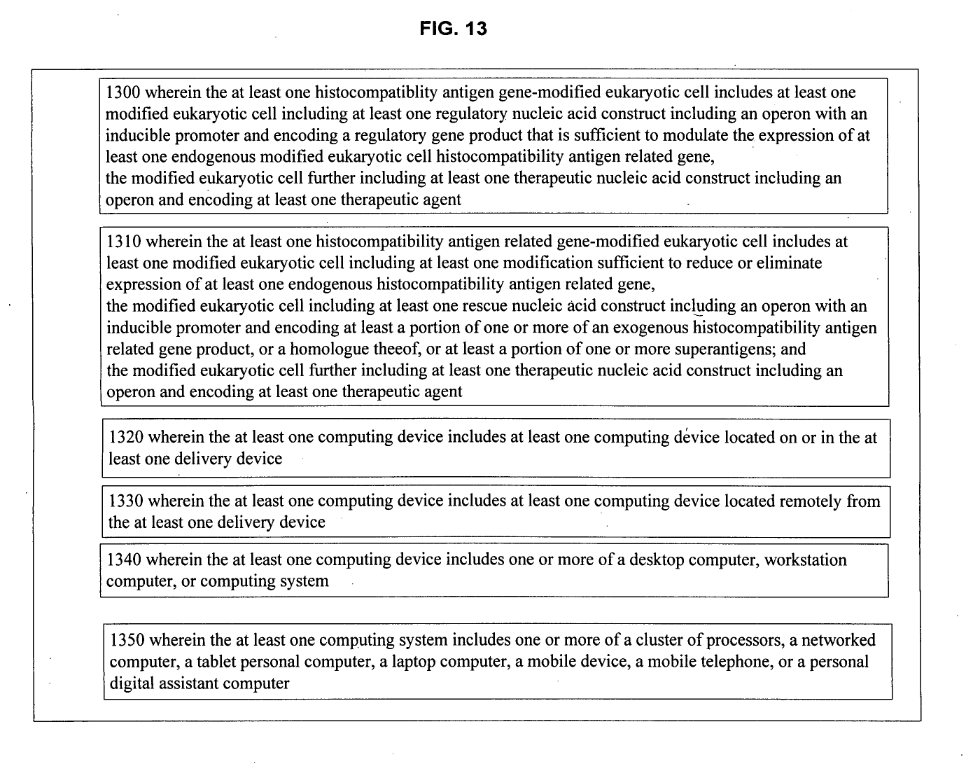

As illustrated in FIG. 13, in an embodiment 1300, the at least one histocompatiblity antigen gene modified eukaryotic cell includes at least one modified eukaryotic cell including at least one regulatory nucleic acid construct including an operon with an inducible promoter and encoding a regulatory gene product that is sufficient to modulate the expression of at least one endogenous modified eukaryotic cell histocompatibility antigen related gene, the modified eukaryotic cell further including at least one therapeutic nucleic acid construct including an operon and encoding at least one therapeutic agent. In an embodiment 1310, the at least one histocompatibility antigen related gene modified eukaryotic cell includes at least one modified eukaryotic cell including at least one modification sufficient to reduce or eliminate expression of at least one endogenous histocompatibility antigen related gene, the modified eukaryotic cell including at least one rescue nucleic acid construct including an operon with an inducible promoter and encoding at least a portion of one or more of an exogenous histocompatibility antigen related gene product, or a homologue thereof, or at least a portion of one or more superantigens; and the modified eukaryotic cell further including at least one therapeutic nucleic acid construct including an operon and encoding at least one therapeutic agent. In an embodiment 1320, the at least one computing device includes at least one computing device located on or in the at least one delivery device. In an embodiment 1330, the at least one computing device includes at least one computing device located remotely from the at least one delivery device. In an embodiment 1340, the at least one computing device includes one or more of a desktop computer, workstation computer, or computing system. In an embodiment 1350, the at least one computing system includes one or more of a cluster of processors, a networked computer, a tablet personal computer, a laptop computer, a mobile device, a mobile telephone, or a personal digital assistant computer.

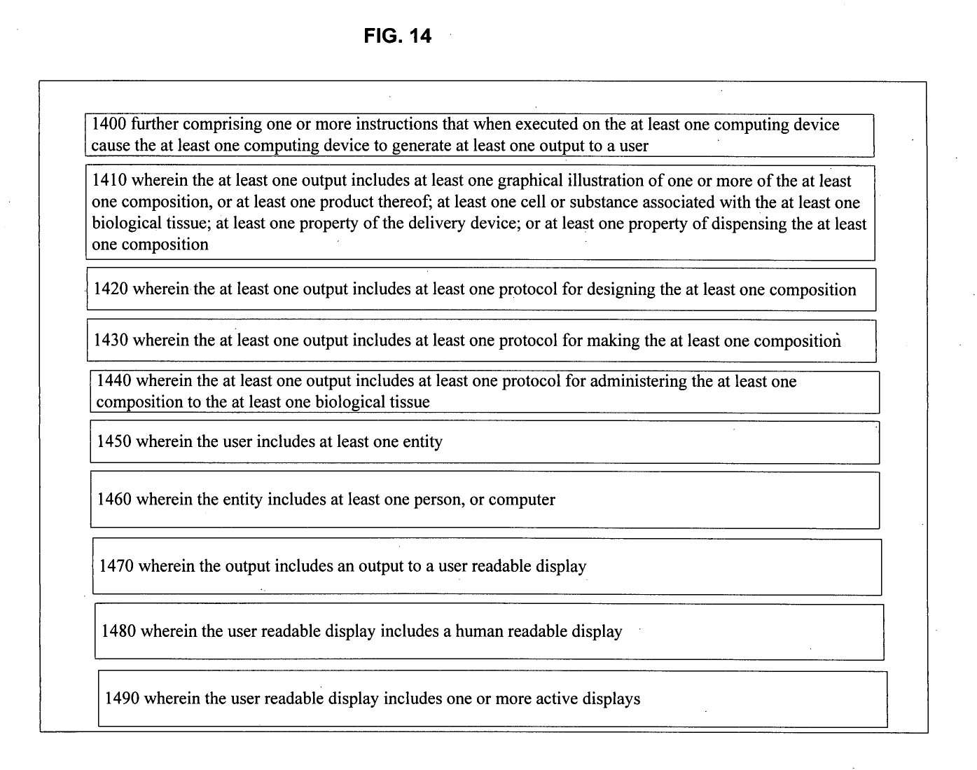

As illustrated in FIG. 14, in an embodiment 1400, the system further comprises one or more instructions that when executed on the at least one computing device cause the at least one computing device to generate at least one output to a user. In an embodiment 1410, the at least one output includes at least one graphical illustration of one or more of the at least one composition, or at least one product thereof; at least one cell or substance associated with the at least one biological tissue; at least one property of the delivery device; or at least one property of dispensing the at least one composition. In an embodiment 1420, the at least one output includes at least one protocol for designing the at least one composition. In an embodiment 1430, the at least one output includes at least one protocol for making the at least one composition. In an embodiment 1440, the at least one output includes at least one protocol for administering the at least one composition to the at least one biological tissue. In an embodiment 1450, the user includes at least one entity. In an embodiment 1460, the entity includes at least one person, or computer. In an embodiment 1470, the output includes an output to a user readable display. In an embodiment 1480, the user readable display includes a human readable display. In an embodiment 1490, the user readable display includes one or more active displays.

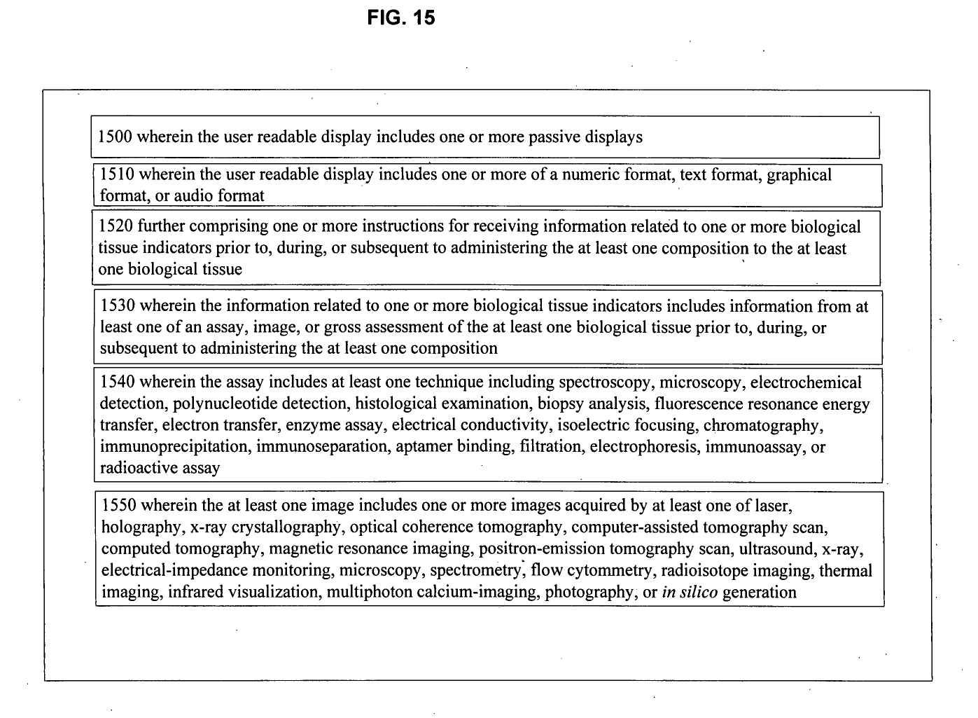

As illustrated in FIG. 15, in an embodiment 1500, the user readable display includes one or more passive displays. In an embodiment 1510, the user readable display includes one or more of a numeric format, text format, graphical format, or audio format. In an embodiment 1520, the system further comprises one or more instructions for receiving information related to one or more biological tissue indicators prior to, during, or subsequent to administering the at least one composition to the at least one biological tissue. In an embodiment 1530, the information related to one or more biological tissue indicators includes information from at least one of an assay, image, or gross assessment of the at least one biological tissue prior to, during, or subsequent to administering the at least one composition. In an embodiment 1540, the assay includes at least one technique including spectroscopy, microscopy, electrochemical detection, polynucleotide detection, histological examination, biopsy analysis, fluorescence resonance energy transfer, electron transfer, enzyme assay, electrical conductivity, isoelectric focusing, chromatography, immunoprecipitation, immunoseparation, aptamer binding, filtration, electrophoresis, immunoassay, or radioactive assay. In an embodiment 1550, the at least one image includes one or more images acquired by at least one of laser, holography, x-ray crystallography, optical coherence tomography, computer-assisted tomography scan, computed tomography, magnetic resonance imaging, positron-emission tomography scan, ultrasound, x-ray, electrical-impedance monitoring, microscopy, spectrometry, flow cytommetry, radioisotope imaging, thermal imaging, infrared visualization, multiphoton calcium-imaging, photography, or in silico generation.

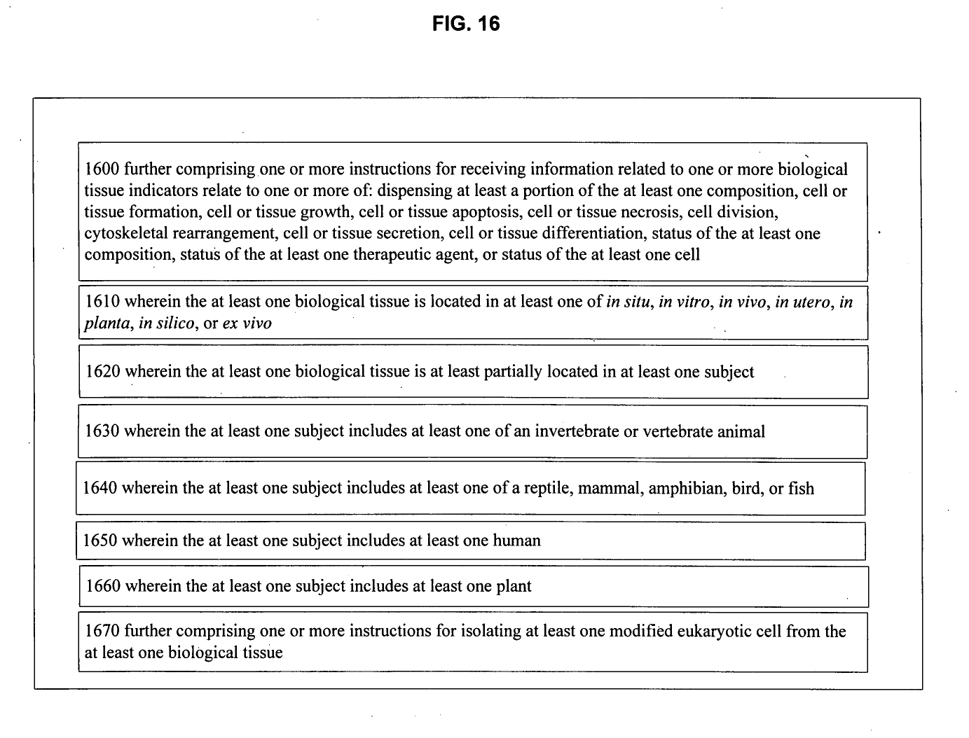

As illustrated in FIG. 16, in an embodiment 1600, the system further comprises one or more instructions for receiving information related to one or more biological tissue indicators relate to one or more of: dispensing at least a portion of the at least one composition, cell or tissue formation, cell or tissue growth, cell or tissue apoptosis, cell or tissue necrosis, cell division, cytoskeletal rearrangement, cell or tissue secretion, cell or tissue differentiation, status of the at least one composition, status of the at least one therapeutic agent, or status of the at least one cell. In an embodiment 1610, the at least one biological tissue is located in at least one of in situ, in vitro, in vivo, in utero, in planta, in silico, or ex vivo. In an embodiment 1620, the at least one biological tissue is at least partially located in at least one subject. In an embodiment 1630, the at least one subject includes at least one of an invertebrate or vertebrate animal. In an embodiment 1640, the at least one subject includes at least one of a reptile, mammal, amphibian, bird, or fish. In an embodiment 1650, the at least one subject includes at least one human. In an embodiment 1660, the at least one subject includes at least one plant. In an embodiment 1670, the system further comprises one or more instructions for isolating at least one modified eukaryotic cell from the at least one biological tissue.

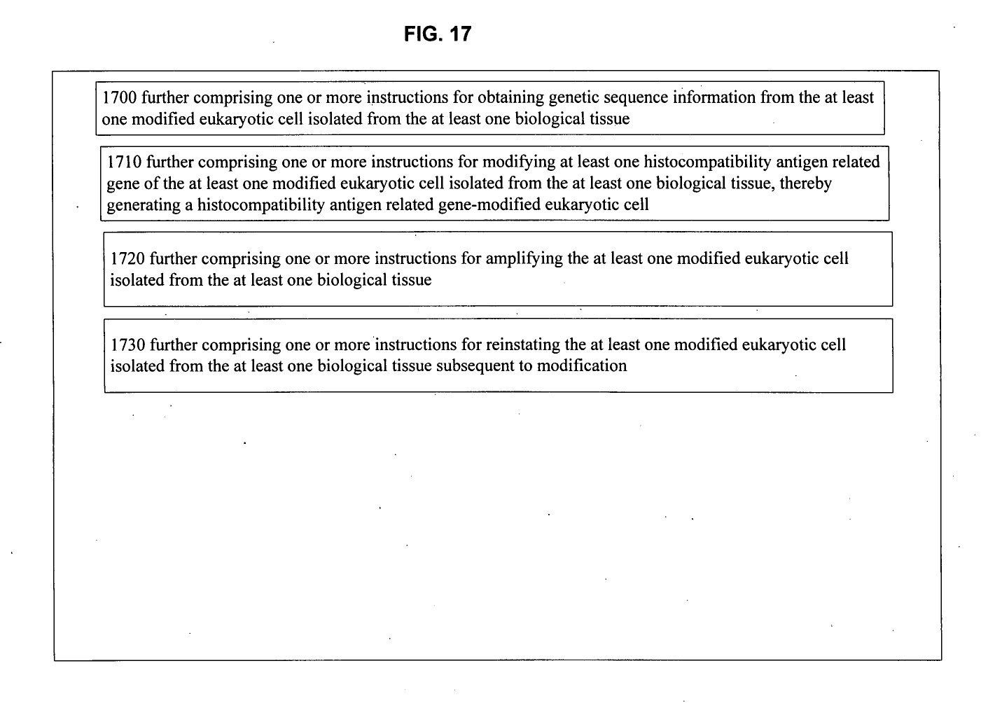

As illustrated in FIG. 17, in an embodiment 1700, the system further comprises one or more instructions for obtaining genetic sequence information from the at least one modified eukaryotic cell isolated from the at least one biological tissue. In an embodiment 1710, the system further comprises one or more instructions for modifying at least one histocompatibility antigen related gene of the at least one modified eukaryotic cell isolated from the at least one biological tissue, thereby generating a histocompatibility antigen related gene modified eukaryotic cell. In an embodiment 1720, the system further comprises one or more instructions for amplifying the at least one modified eukaryotic cell isolated from the at least one biological tissue. In an embodiment 1730, the system further comprises one or more instructions for reinstating the at least one modified eukaryotic cell isolated from the at least one biological tissue subsequent to modification.

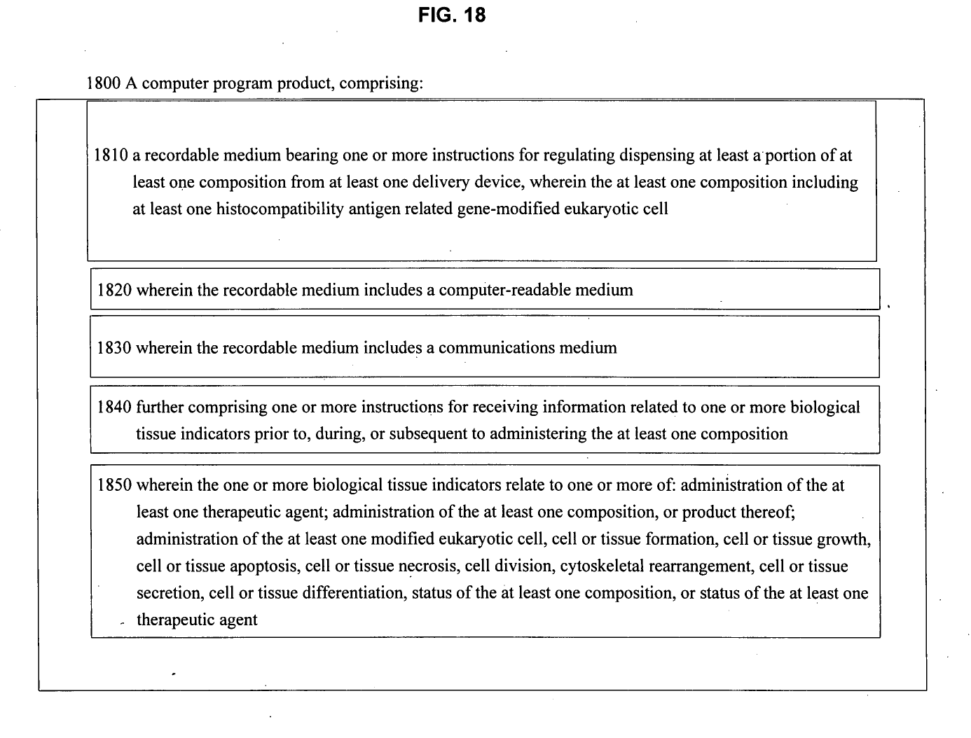

As illustrated in FIG. 18, a computer program product 1800, comprises: 1810 a recordable medium bearing one or more instructions for regulating dispensing at least a portion of at least one composition from at least one delivery device, wherein the at least one composition including at least one histocompatibility antigen related gene modified eukaryotic cell. In an embodiment 1820, the recordable medium includes a computer-readable medium. In an embodiment 1830, the recordable medium includes a communications medium. In an embodiment 1840, the computer program product further comprises one or more instructions for receiving information related to one or more biological tissue indicators prior to, during, or subsequent to administering the at least one composition. In an embodiment 1850, the one or more biological tissue indicators relate to one or more of: administration of the at least one therapeutic agent; administration of the at least one composition, or product thereof; administration of the at least one modified eukaryotic cell, cell or tissue formation, cell or tissue growth, cell or tissue apoptosis, cell or tissue necrosis, cell division, cytoskeletal rearrangement, cell or tissue secretion, cell or tissue differentiation, status of the at least one composition, or status of the at least one therapeutic agent.

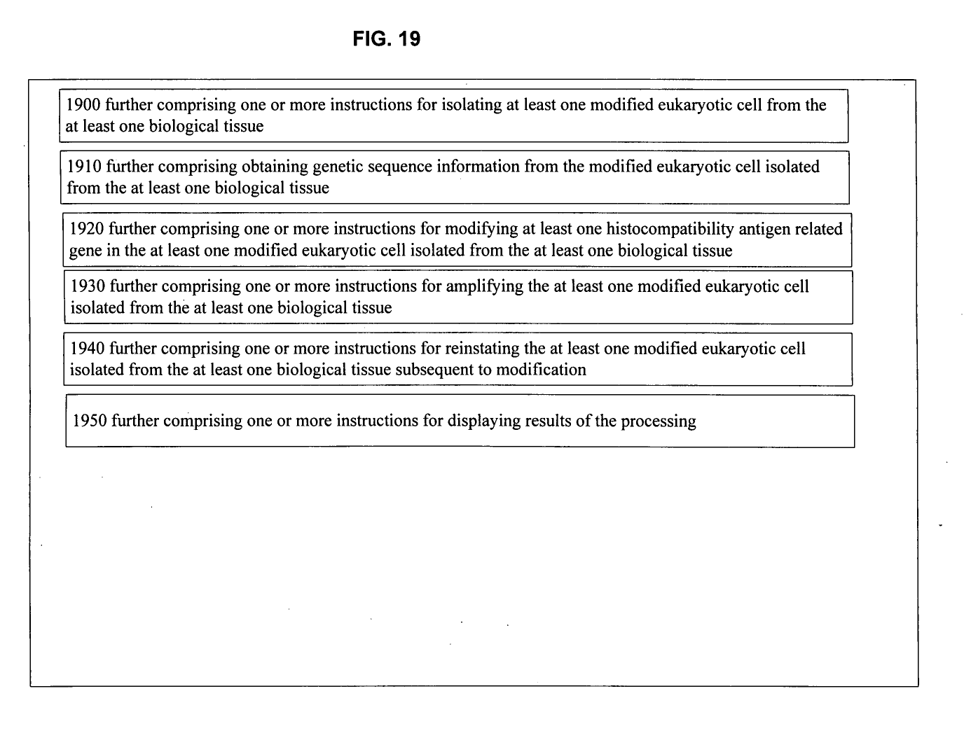

As illustrated in FIG. 19, the computer program product further comprises one or more instructions for isolating at least one modified eukaryotic cell from the at least one biological tissue. In an embodiment 1910, the computer program product further comprises obtaining genetic sequence information from the modified eukaryotic cell isolated from the at least one biological tissue. In an embodiment 1920, the computer program product further comprises one or more instructions for modifying at least one histocompatibility antigen related gene in the at least one modified eukaryotic cell isolated from the at least one biological tissue. In an embodiment 1930, the computer program product further comprises one or more instructions for amplifying the at least one modified eukaryotic cell isolated from the at least one biological tissue. In an embodiment 1940, the computer program product further comprises one or more instructions for reinstating the at least one modified eukaryotic cell isolated from the at least one biological tissue subsequent to modification. In an embodiment 1950, the computer program product further comprises one or more instructions for displaying results of the processing.

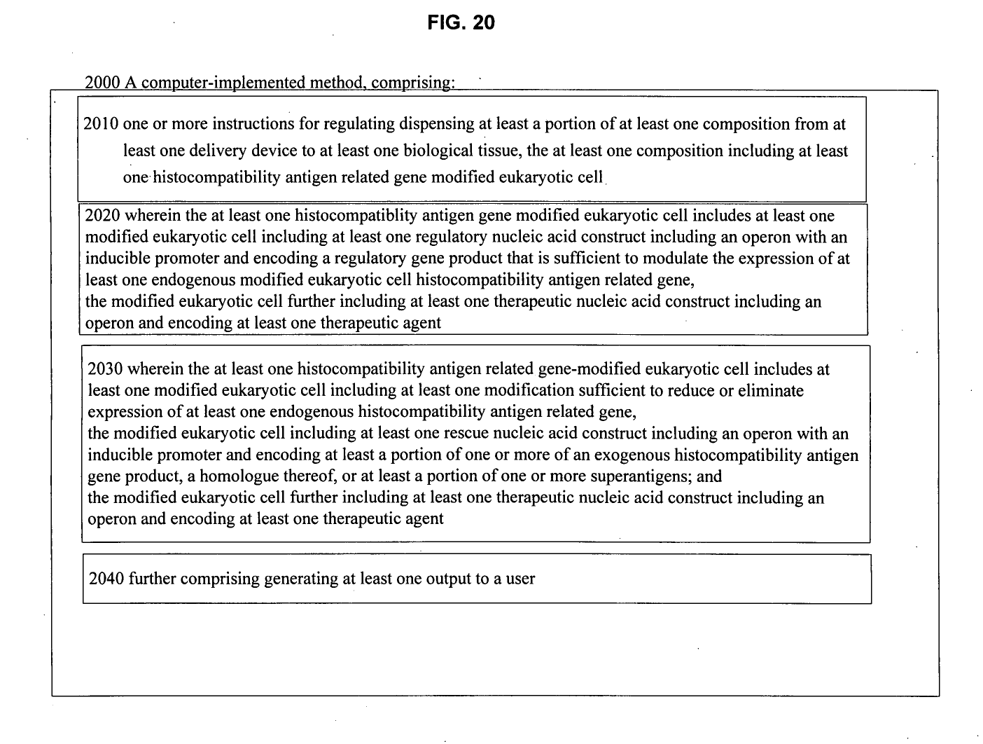

As illustrated in FIG. 20, a computer-implemented method 2000, comprises: 2010 one or more instructions for regulating dispensing at least a portion of at least one composition from at least one delivery device to at least one biological tissue, the at least one composition including at least one histocompatibility antigen related gene modified eukaryotic cell. In an embodiment 2020, the at least one histocompatiblity antigen gene modified eukaryotic cell includes at least one modified eukaryotic cell including at least one regulatory nucleic acid construct including an operon with an inducible promoter and encoding a regulatory gene product that is sufficient to modulate the expression of at least one endogenous modified eukaryotic cell histocompatibility antigen related gene, the modified eukaryotic cell further including at least one therapeutic nucleic acid construct including an operon and encoding at least one therapeutic agent.

In an embodiment 2030, the at least one histocompatibility antigen related gene modified eukaryotic cell includes at least one modified eukaryotic cell including at least one modification sufficient to reduce or eliminate expression of at least one endogenous histocompatibility antigen related gene, the modified eukaryotic cell including at least one rescue nucleic acid construct including an operon with an inducible promoter and encoding at least a portion of one or more of an exogenous histocompatibility antigen gene product, a homologue thereof, or at least a portion of one or more superantigens; and the modified eukaryotic cell further including at least one therapeutic nucleic acid construct including an operon and encoding at least one therapeutic agent. In an embodiment 2040, the computer-implemented method further comprising generating at least one output to a user.

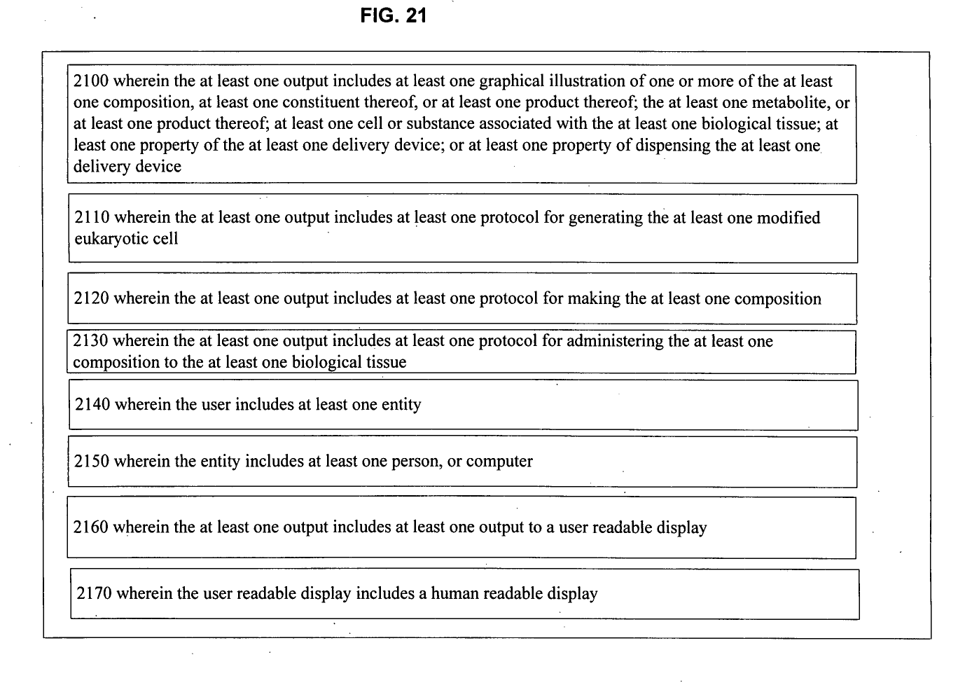

As illustrated in FIG. 21, in an embodiment 2100, the at least one output includes at least one graphical illustration of one or more of the at least one composition, at least one constituent thereof, or at least one product thereof; the at least one metabolite, or at least one product thereof; at least one cell or substance associated with the at least one biological tissue; at least one property of the at least one delivery device; or at least one property of dispensing the at least one delivery device. In an embodiment 2110, the at least one output includes at least one protocol for generating the at least one modified eukaryotic cell. In an embodiment 2120, the at least one output includes at least one protocol for making the at least one composition. In an embodiment 2130, the at least one output, includes at least one protocol for administering the at least one composition to the at least one biological tissue. In an embodiment 2140, the user includes at least one entity. In an embodiment 2150, the entity includes at least one person, or computer. In an embodiment 2160, the at least one output includes at least one output to a user readable display. In an embodiment 2170, the user readable display includes a human readable display.

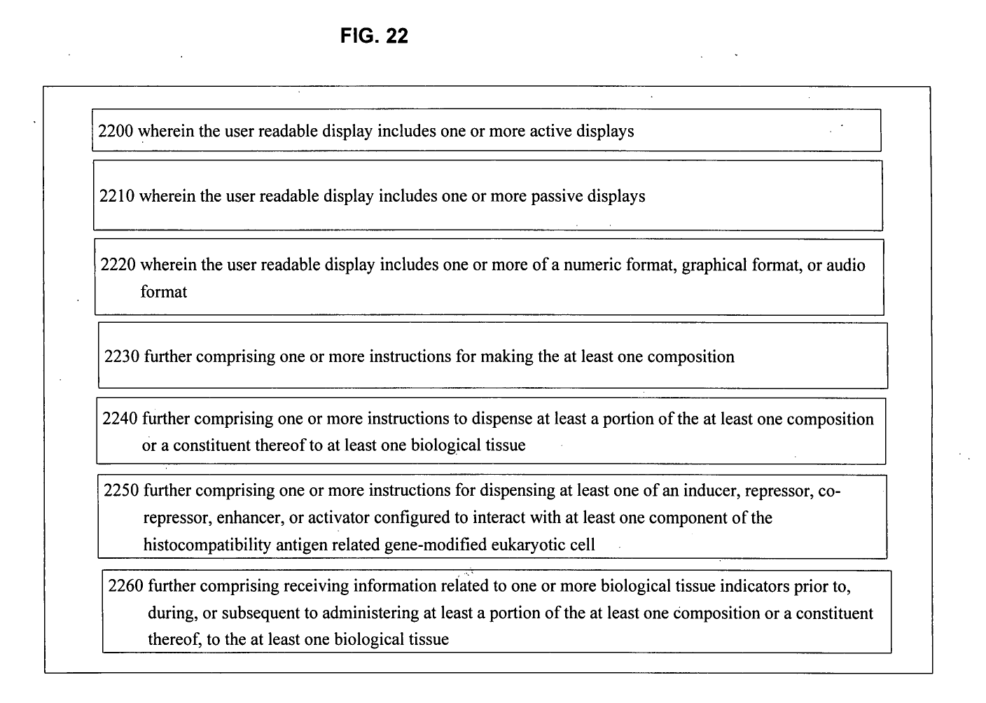

As illustrated in FIG. 22, in an embodiment 2200, the user readable display includes one or more active displays. In an embodiment 2210, the user readable display includes one or more passive displays. In an embodiment 2220, the user readable display includes one or more of a numeric format, graphical format, or audio format. In an embodiment 2230, the computer-implemented method further comprises one or more instructions for making the at least one composition. In an embodiment 2240, the computer-implemented method further comprises one or more instructions to dispense at least a portion of the at least one composition or a constituent thereof to at least one biological tissue. In an embodiment 2250, the computer-implemented method further comprises one or more instructions for dispensing at least one of an inducer, repressor, co-repressor, enhancer, or activator configured to interact with at least one component of the histocompatibility antigen related gene modified eukaryotic cell. In an embodiment 2260, the computer-implemented method further comprises receiving information related to one or more biological tissue indicators prior to, during, or subsequent to administering at least a portion of the at least one composition or a constituent thereof, to the at least one biological tissue.

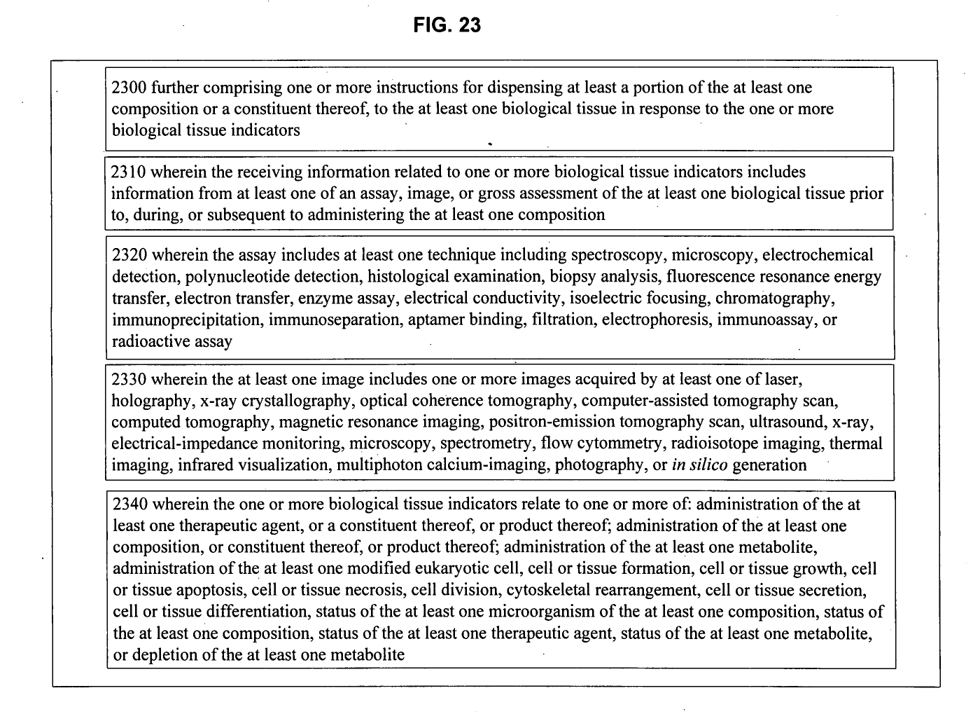

As illustrated in FIG. 23, in an embodiment 2300, the computer-implemented method further comprises one or more instructions for dispensing at least a portion of the at least one composition or a constituent thereof, to the at least one biological tissue in response to the one or more biological tissue indicators. In an embodiment 2310, the receiving information related to one or more biological tissue indicators includes information from at least one of an assay, image, or gross assessment of the at least one biological tissue prior to, during, or subsequent to administering the at least one composition. In an embodiment 2320, the assay includes at least one technique including spectroscopy, microscopy, electrochemical detection, polynucleotide detection, histological examination, biopsy analysis, fluorescence resonance energy transfer, electron transfer, enzyme assay, electrical conductivity, isoelectric focusing, chromatography, immunoprecipitation, immunoseparation, aptamer binding, filtration, electrophoresis, immunoassay, or radioactive assay. In an embodiment 2330, the at least one image includes one or more images acquired by at least one of laser, holography, x-ray crystallography, optical coherence tomography, computer-assisted tomography scan, computed tomography, magnetic resonance imaging, positron-emission tomography scan, ultrasound, x-ray, electrical-impedance monitoring, microscopy, spectrometry, flow cytommetry, radioisotope imaging, thermal imaging, infrared visualization, multiphoton calcium-imaging, photography, or in silico generation. In an embodiment 2340, the one or more biological tissue indicators relate to one or more of: administration of the at least one therapeutic agent, or a constituent thereof, or product thereof; administration of the at least one composition, or constituent thereof, or product thereof; administration of the at least one metabolite, administration of the at least one modified eukaryotic cell, cell or tissue formation, cell or tissue growth, cell or tissue apoptosis, cell or tissue necrosis, cell division, cytoskeletal rearrangement, cell or tissue secretion, cell or tissue differentiation, status of the at least one microorganism of the at least one composition, status of the at least one composition, status of the at least one therapeutic agent, status of the at least one metabolite, or depletion of the at least one metabolite.

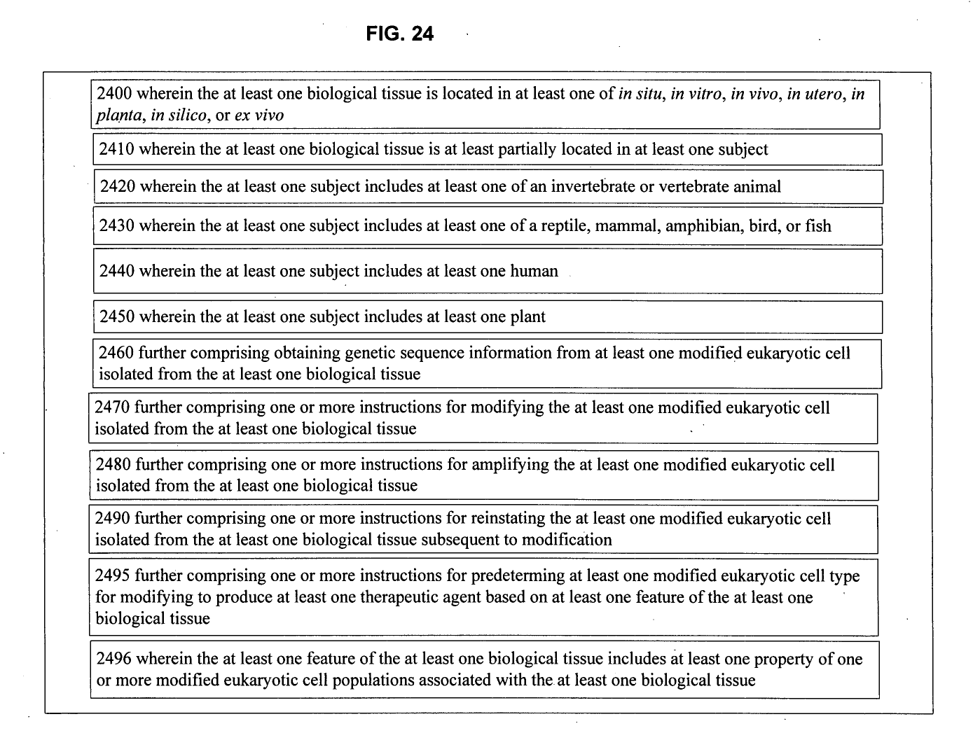

As illustrated in FIG. 24, in an embodiment 2400, the at least one biological tissue is located in at least one of in situ, in vitro, in vivo, in utero, in planta, in silico, or ex vivo. In an embodiment 2410, the at least one biological tissue is at least partially located in at least one subject. In an embodiment 2420, the at least one subject includes at least one of an invertebrate or vertebrate animal. In an embodiment 2430, the at least one subject includes at least one of a reptile, mammal, amphibian, bird, or fish. In an embodiment 2440, the at least one subject includes at least one human. In an embodiment 2450, the at least one subject includes at least one plant. In an embodiment 2460, the computer-implemented method further comprises obtaining genetic sequence information from at least one modified eukaryotic cell isolated from the at least one biological tissue. In an embodiment 2470, the computer-implemented method further comprises one or more instructions for modifying the at least one modified eukaryotic cell isolated from the at least one biological tissue. In an embodiment 2480, the computer-implemented method further comprises one or more instructions for amplifying the at least one modified eukaryotic cell isolated from the at least one biological tissue. In an embodiment 2490, the computer-implemented method, further comprises one or more instructions for reinstating the at least one modified eukaryotic cell isolated from the at least one biological tissue subsequent to modification. In an embodiment 2495, the computer-implemented method further comprises one or more instructions for predetermining at least one modified eukaryotic cell type for modifying to produce at least one therapeutic agent based on at least one feature of the at least one biological tissue. In an embodiment 2496, the at least one feature of the at least one biological tissue includes at least one property of one or more modified eukaryotic cell populations associated with the at least one biological tissue.

### PROPHETIC EXAMPLES

### Prophetic Example 1

### MHC-Less Cells in Organ Transplantation

A patient infected with a hepatitis C virus and presenting with a fibrotic liver is treated with unmatched, allogeneic donor hepatocytes engineered to block the presentation of Major Histocompatibility Class I (MHC I) proteins on their cell surface and to produce and secrete the cytokine interferon lambda-3 (IFN λ3). Allogeneic donor hepatocytes are transfected with a lentiviral expression vector that directs the expression of a microRNA (miRNA) that inhibits beta2-microglobulin (β₂M) protein translation and blocks MHC I assembly and presentation on the cell surface. The lentiviral expression vector also directs the expression of IFN λ3, a cytokine that evokes an antiviral response from the hepatocytes. The genetically engineered hepatocytes are injected into the patient to replace fibrotic liver sections that have been resected. The inhibition of MHC I production in engrafted hepatocytes is controlled by a regulatory module and an effector molecule, doxycycline. In the event that the engineered hepatocytes must be eradicated, doxycycline is administered to repress expression of the miRNA, thereby allowing expression of β₂M and MHC I on the cell surface and evoking an alloreactive immune response.

Hepatocytes are obtained from a liver donor who died in a motorcycle accident. The donor liver cells are allogeneic to the recipient's cells (i.e. nonidentical MHC I alleles are present in at least one locus of the MHC I gene complex). For example, the donor may have a HLA-A2 gene and a HLA-A3 gene versus the recipient who may have a HLA-A1 gene and a HLA-A4 gene. The donor liver tissue is processed to obtain hepatocytes. Methods to obtain viable hepatocytes from liver tissue are known in the art (see e.g., U.S. Pat. No. 7,351,584, which is incorporated herein by reference). Isolated hepatocytes are cultured in 100-mm diameter sterile petri dishes, or an equivalent thereof, with 10 mL of Waymouth's 752/1 medium, pH 7.28. Medium is preferably supplemented with the following: 2.24 g/L sodium bicarbonate, 2.38 g/L HEPES buffer, 11.2 mg/L alanine, 12.8 mg/L serine, 24 mg/L asparagine, 0.3 mL heptanoic acid, 5 mg/L linoleic acid, 0.175 mg/L aminol evulinic acid, 5 mg/L insulin, 5 40 mg/L transferrin, 5 μl/L selenous acid, 39.2 μg/L dexamethasone, 0.25 mg/L amphotericin B, 84 mg/L gentamicin sulfate, 84 mg/L amikacin sulfate, 100 U/mL penicillin G sodium, and 100 mg/L streptomycin sulfate (media components are available from Sigma Aldrich Chemicals Inc., St. Louis, Mo.). The hepatocytes are cultured at approximately 10⁶ cells/ml in an incubator at 37° C. with an atmosphere of 5% CO₂ in air, prior to transfection with a lentiviral expression vector.

A lentiviral expression vector is constructed that encodes a β₂M microRNA (miRNA) and contains tetracycline regulatory elements. Lentiviral vectors suitable for in vitro delivery of miRNA and target genes to primary cells and nondividing cells are known in the art. (See e.g., Technical Manual: “BLOCK-iT™ Lentiviral miR RNAi Expression System”, Version A, Jun. 29, 2005 available from Invitrogen, Carlsbad, Calif. 92008 which is incorporated herein by reference.) A DNA sequence encoding a miRNA for β₂M is cloned into a plasmid-based expression vector containing required elements for packaging the expression construct into virions. The plasmid is combined with a packaging mixture and transfected into a 293FT cell line to produce a recombinant nonreplicating lentivirus. Lentiviral stocks with a titer of approximately 10⁵ to 10⁷ transducing units/ml are sufficient to transduce 10⁶-10⁸ hepatocytes at a multiplicity of infection of 1.0.

To determine the titer of the lentivirus stock, serial ten-fold dilutions of the stock are applied to a hepatic cell line and the number of transduced cells is counted after growth in blasticidin. Blasticidin is used to select stably transduced cells via a corresponding drug-resistance marker on the lentiviral expression vector. Methods to design and synthesize double stranded DNA molecules that encode miRNA precursors are known in the art (see e.g., Technical Manual: Invitrogen Ibid. and Zeng et al, Molecular Cell 9: 1327-1333, (2002), which are incorporated herein by reference). Expression of the β₂M miRNA is controlled by a cytomegalovirus promoter with an adjacent tetracycline response element. A Tet-Off® Advanced system is used to regulate the expression of the β₂M miRNA (see e.g., Product Sheet: “Tet-On® and Tet-Off® Advanced Inducible Gene Expression Systems” available from Clontech Laboratories, Inc. Mountain View, Calif.). The expression system actively transcribes the β₂M miRNA in the absence of doxycycline (a stable analog of tetracycline) and ceases transcription of the β₂M miRNA when doxycycline is administered.

Hepatocytes are transfected with the lentiviral vector encoding a β₂M miRNA that is regulated by doxycycline. The miRNA inhibits β₂M translation and reduces the amount of β₂M protein, which ultimately decreases the amount of MHC I presented on the hepatocyte cell surface (see e.g., Hill et al., J. Biol. Chem. 278: 5630-5638, (2003), which is incorporated herein by reference). To protect hepatocyte recipients from uncontrolled proliferation of the transfected hepatocytes or other potential adverse events, hepatocyte expression of the β₂M miRNA may be inhibited by the administration of doxycycline to the hepatocyte recipient. Without β₂M miRNA to block β₂M translation, its protein levels are restored and allogeneic MHC I is expressed on the cell surface of the hepatocytes, thus making them vulnerable to alloreactive T cells. An alloreactive cell-mediated immune response eliminates the transfected hepatocytes (see e.g., Game and Lechler, Transplant Immunol. 10: 101-108, 2002, which is incorporated herein by reference).

To protect donor hepatocytes from infection by HCV, the lentiviral expression vector also contains an operon that directs the expression of an antiviral cytokine, IFNλ3 (see e.g., Thomas et al., Nature 461: 798-801, 2009, which is incorporated herein by reference). The IFNλ3 gene, denoted IL28B, is introduced into the lentiviral expression construct and constitutively expressed by hepatocytes transfected with the lentiviral vector. Transfected hepatocytes secrete IFNλ3, and evoke an antiviral response in the recipient's liver cells as well as in transfected donor hepatocytes.

Allogeneic hepatocytes stably transduced with a lentiviral vector encoding β₂M miRNA, IFNλ3 and tetracycline regulatory elements are transplanted into a patient with a fibrotic liver who is infected with HCV. Methods to transplant hepatocytes into humans with liver failure are known in the art (see e.g., Fitzpatrick et al., J. of Internal Med. 266: 339-357, 2009 and U.S. Patent App. Pub. No. 20080233088, each of which is incorporated herein by reference). Approximately 5×10¹° to 5×10¹² hepatocytes are administered to the patient. The transduced hepatocytes may be administered to the liver, for example into the hepatic portal vein, intravenously, under the kidney capsule, or into the spleen. No more than 10⁸ hepatocytes per kg of body weight may be transplanted via the portal vein as a single procedure.

Engraftment and proliferation of the hepatocyte graft are determined by biopsy of the graft (e.g., liver biopsy) and immunohistochemistry studies to identify hepatocytes expressing lentiviral proteins, IFNλ3 and specific MHC I alleles derived from the recipient. Methods to identify engrafted cells are known in the art (see e.g., U.S. Patent Application No. 20080233088, Ibid., incorporated herein by reference). Also the serum level of IFNλ3 is measured to assess production of IFNλ3 by the engrafted hepatocytes. Biochemical parameters are monitored to evaluate the function of the engrafted liver. For example, serum or plasma from the patient is analyzed to determine albumin, alanine transaminase, aspartate transaminase, alkaline phosphatase and total bilirubin levels. Patient values are compared to normal ranges previously established for each of these analytes. An additional measure of hepatocyte engraftment and proliferation is obtained by magnetic resonance imaging. Images of the liver post-transplantation are compared to images of the resected liver prior to hepatocyte transplantation. Results of the liver function tests, biopsy, IFNλ3 assay, and magnetic resonance imaging, are compiled to evaluate engraftment and proliferation of the transduced hepatocytes. If excessive proliferation is apparent, the patient may be given doxycycline to restore expression of allogeneic MHC I and induce an alloreactive immune response to eliminate the transduced hepatocytes.

### Prophetic Example 2

### MHC-Less Cells for Delivery of Therapeutic Agent

A patient with progressive multiple sclerosis is treated with engineered oligodendrocyte lineage cells (OLC) that have reduced expression of Major Histocompatibility Class I (MHC I) proteins on their cell surface, in order to avoid immune rejection of the transplanted cells. The OLC are also engineered to express a therapeutic protein that is anti-inflammatory and that promotes re-myelination of lesions in the myelin sheath. The engineered OLC also contain a suicide mechanism that can be activated by the administration of a prodrug, ganciclovir, in the event of uncontrolled proliferation or other adverse events associated with the OLC.

A patient with secondary progressive multiple sclerosis is treated by transplantation of OLC that derive from embryonic stem cells (ESC) obtained from an allogeneic donor. The OLC are modified to express an adenovirus gene encoding glycoprotein 19 (gp19), which prevents expression of MHC I on the cell surface, from early transcription region 3 (E3). The OLC are also genetically engineered to secrete an anti-inflammatory cytokine, interleukin-10 (IL-10), that promotes re-myelination, and to express a herpes simplex virus thymidine kinase (HSV-TK) gene that converts ganciclovir into a cytotoxic metabolite.

Oligodendrocyte lineage cells are derived from ESC and modified with an adenovirus gene, E3 gp19, that down-regulates the transport of MHC I proteins to the cell surface. Methods to derive OLC from ESC are known in the art (see e.g., U.S. Patent App. Pub. No. 2009/0232779; and Glaser et al., FASEB J. 19: 112-114, (2004), each of which is incorporated herein by reference). Primate ESCs are obtained from blastocysts or fetal or embryonic tissue, and may be primary cultures or established cell lines. Established human ESC lines are available from WiCell Research Institute, Madison, Wis. and the American Type Culture Collection, Manassas, Va. ESC are differentiated to OLC by culture in a series of different culture conditions with different medium additives.

For example, ESC cells are sequentially cultured as follows:
Day 1) in suspension in TR* medium containing 4 ng/mL basic fibroblast growth factor (bFGF);
Day 2) TR medium containing 10 μM retinoic acid and 4 ng/mL bFGF;
Day 3-10) GRMA medium containing 10 μM retinoic acid;
Day 11-15) GRM medium containing 2 ng/mL bFGF, 20 ng/mL epidermal growth factor (EGF);
Day 15-28) GRM medium containing 20 ng/mL EGF;
Day 28) on Matrigel® in GRM medium containing 20 ng/mL EGF;
Day 29-35) on Matrigel® in GRM medium containing 20 ng/mL EGF;
Day 35-36) on poly-L-lysine-Laminin in GRM medium containing 20 ng/mL EGF, 2 ng/mL bFGF;
Day 37-41) in GRM medium
*TR medium contains 50% GRM and 50% conditioned media derived from mouse embryo fibroblast cultures.
̂GRM medium contains DMEM:F12, B27 supplement, insulin, progesterone, putrescine, sodium selenite, transferrin and triiodothyronine. All components are available from Sigma-Aldrich Chemical Co., St. Louis, Mo. and/or Life Technologies Corp., Carlsbad, Calif.)

The differentiated cells are tested by immunocytochemistry using a series of antibodies reactive with OLC. The cells are fixed with 4% paraformaldehyde, washed and then incubated with antibodies specific for galactocerebroside (Gal C), A2B5, polysialylated neural cell adhesion molecule (PSA-NCAM) and nestin (Antibodies are available from Chemicon/Millipore Corp., Billerica, Mass.). Approximately 95% of the differentiated cells are positive for Gal C, a marker for early oligodendrocytes, and approximately 80% to 95% of the cells may test positive for A2B5, PSA-NCAM, and nestin, indicating cells of oligodendrocyte lineage.

Oligodendrocyte lineage cells are transfected with mammalian cell expression vectors that encode 1) the E3 gp19 protein derived from adenovirus, 2) the anti-inflammatory cytokine IL-10, and 3) the HSV-TK gene. Methods to express viral genes in mammalian primary cells or cell lines are known in the art (see e.g., U.S. Pat. No. 6,491,909, which is incorporated herein by reference). A DNA fragment encoding gp19, from the early transcription region 3 of Adenovirus 2, is inserted into an expression plasmid containing a tetracycline response element and a minimal cytomegalovirus promoter. A Tet-Off® Advanced system is used to regulate the expression of gp19 (see e.g., Product Sheet: “Tet-On® and Tet-Off® Advanced Inducible Gene Expression Systems” available from Clontech Laboratories, Inc. Mountain View, Calif.). The expression system actively transcribes the gp19 mRNA in the absence of doxycycline (a stable tetracycline analog) and ceases transcription of the gp19 mRNA when doxycycline is administered.

Expression of gp19 protein in OLC leads to a block in transport of MHC Class I that reduces MHC I expression on the cell surface (see e.g., U.S. Pat. No. 6,491,909 Ibid., incorporated herein by reference). OLC that express gp19 are tested for the presence, or lack thereof, of cell surface MHC I proteins using immunohistochemistry and flow cytometry. Antibodies specific for HLA-A, HLA-B and HLA-C(HLA-A, -B, -C antibodies are available from BioLegend, San Diego, Calif.) are used to compare MHC I levels on cells that are expressing gp19 (no doxycycline) to cells not expressing gp19 (with doxycycline).

Oligodendrocyte lineage cells are also transfected with a mammalian cell expression vector that encodes a therapeutic protein, IL-10, and a suicide gene, HSV-TK. A bicistronic expression plasmid is constructed to express human IL-10 and HSV-TK. The bicistronic construct directs the expression of IL-10 and HSV-TK on a single transcript controlled by the EF-1 promoter. Bicistronic vectors for mammalian cell expression are known in the art (see e.g., the Product Sheet entitled, “BICEP Vectors for Bicistronic Expression” available from Sigma-Aldrich, St. Louis, Mo. which is incorporated herein by reference). OLC are engineered to produce IL-10 constitutively by transfection with a bicistronic vector containing an EF-1 promoter and the human IL-10 gene (available from the American Type Culture Collection, Manassas, Va.). OLC engineered to produce IL-10 are injected intravenously and into the brain (specifically the corpus callosum) of the MS patient where IL-10 produced by OLC migrating to the brain and the spinal tract of the MS patient reduces inflammation and promotes re-myelination of axons. For example, IL-10 can reduce the numbers of CD45⁺ and CD68⁺ inflammatory cells that infiltrate the CNS, and also promote re-myelination of demyelinated axons in the spinal cord (see e.g., Yang et al., J. Clin. Invest. 119: 3678-3691, (2009), which is incorporated herein by reference). Magnetic resonance imaging with gadolinium enhancement is used to evaluate re-myelination and the number of plaques (lesions) present in the brain of the MS patient. Flow cytometry of cerebral-spinal fluid (CSF) and of peripheral blood is used to evaluate the presence of inflammatory cells (e.g., macrophage, monocytes, granulocytes, T cells, B cells,). Also, immunoassay for IL-10 levels in peripheral blood and in CSF indicates the level of anti-inflammatory protein produced by the OLC in vivo. Kits for immunoassay of IL-10 are available from Becton Dickinson, Franklin Lakes, N.J.

To stop uncontrolled proliferation of the engineered OLC or other adverse effects, a suicide gene, HSV-TK, is also expressed from the bicistronic expression vector. Methods to express the HSV-TK gene and to activate a cytotoxic prodrug such as ganciclovir are known in the art (see e.g., U.S. Pat. No. 6,576,464, which is incorporated herein by reference). The HSV-TK gene is inserted in the bicistronic vector using recombinant DNA methods described in the Product Sheet entitled “BICEP Vectors for Bicistronic Expression” available from Sigma-Aldrich, St. Louis, Mo., which is incorporated herein by reference. To stop the growth of OLC deemed unsafe or part of an adverse event, the OLC expressing HSV-TK are provided with 20 μM ganciclovir (available as Cytovene IV from Roche Laboratories, Nutley, N.J.). Conversion of ganciclovir into a toxic metabolite by OLC cells expressing HSV-TK results in death of the OLC. Cells not expressing HSV-TK are not harmed by ganciclovir. A second level of safeguard is provided by restoration of MHC I expression on the OLC. Providing doxycycline to the MS patient engages the Tet-Off® regulatory system in the OLC and stops transcription of gp19 thus allowing MHC I transport and representation on the cell surface to resume. Expression of allogeneic MHC I on the surface of OLC will elicit an alloreactive immune response that will eliminate the OLC (see e.g., Game and Lechler, Transplant Immunology 10: 101-108, (2002), which is incorporated herein by reference).

### Prophetic Example 3

### MHC-Less Cells for Reducing Graft Rejection

A patient with Non-Hodgkin's Lymphoma (NHL) is treated with a hematopoietic stem cell transplant with allogeneic hematopoietic stem cells (HSC) that are modified to reduce their expression of mismatched HLA antigens and thus avoid allograft rejection, and graft versus host disease (GVHD). HSC are infected with a lentivirus vector encoding microRNA (miRNA) that inhibits the expression of specific donor HLA alleles not shared by the recipient. Production of mismatched HLA-A, -B, -C, -DRBI, and -DQB1 alleles is blocked by the miRNA, and the corresponding HLA proteins are not expressed by the modified donor HSC. The vector also encodes a therapeutic protein, interleukin-21 (IL-21) to promote a graft versus tumor immune response by the HSC.

A patient with NHL who has not responded to first line treatment with chemotherapy and/or biological therapy is treated using modified allogeneic HSC and a non-myeloablative preparative regimen. Donor HSCs are obtained from a haplo-identical donor who shares 5 out of 10 of the patient's FILA genes, for example, a parent, sibling or person unrelated to the patient with a haploid set of HLA-A, -B, -C, -DRBI, and -DQB1 genes identical to those of the recipient. Both recipient and donor DNA are genotyped at high resolution by using a combination of oligonucleotide sequence specific amplification and DNA sequencing to determine the identity of the 10 HLA genes at the HLA-A, -B, -C, -DRBI, and -DQB1 loci. Methods to determine HLA genotypes and HLA antigen expression are known in the art (see e.g., Nowak, Bone Marrow Transplantation 42: s71-s76, (2008), which is incorporated herein by reference). Any allogeneic (i.e. nonidentical) alleles from the donor at the HLA-A, -B, -C -DRBI, and -DQB1 loci are targeted for inhibition with miRNA. Methods to obtain HSC from peripheral blood are known in the art (see e.g., Lane et al., Blood 85: 275-282 (1995), which is incorporated herein by reference). To mobilize HSC the donor is administered granulocyte colony-stimulating factor (G-CSF) 10 μg/kg/day subcutaneously for 4 days followed by leukapheresis on day 5. HSCs are selected using magnetic beads and anti-CD34 antibodies (Magnetic beads, antibodies and protocols are available from Miltenyi Biotec, Bergisch Gladbach, Germany.). Approximately 10⁸ mononuclear CD34⁺ cells are obtained, and modified by infection with a lentiviral expression vector.

A lentiviral expression vector is constructed to encode microRNA (miRNA) specific for allogeneic HLA genes expressed by the donor. For example, a donor may have an allogeneic haplotype containing HLA-A*0101, -Cw*0701, -B*0801, -DRB1*0301 and -DQB1*0201 genes which are non-identical to the recipient's HLA genes. The expression of miRNA targeting the allogeneic HLA genes is controlled by tetracycline responsive regulatory elements in combination with a minimal CMV promoter. Lentiviral vectors for expression of miRNA that target genes in primary cells and nondividing cells are known in the art. (See e.g., Technical Manual: “BLOCK-iT™ Lentiviral miR RNAi Expression System”, Version A, Jun. 29, 2005 available from Invitrogen, Carlsbad, Calif. 92008, which is incorporated herein by reference.) DNA sequences encoding miRNA for allogeneic HLA genes are cloned into a plasmid-based expression vector containing required elements for packaging the expression construct into virions. The plasmid is combined with a packaging mixture and transfected into a 293FT cell line to produce a recombinant nonreplicating lentivirus. Lentiviral stocks with a titer of approximately 10⁵ to 10⁷ transducing units/ml are sufficient to transduce 10⁶-10⁸ HSC at a multiplicity of infection of 1.0. To determine the titer of the lentivirus stock, serial ten-fold dilutions of the stock are applied to a hematopoietic cell line, and the number of transduced cells is counted after growth in blasticidin. Blasticidan resistance marker on the lentiviral expression vector selects stably transduced cells. Expression of miRNA specific for the allogeneic HLA-A*0101, -Cw*0701, -B*0801, -DRB1*0301 and -DQB1*0201 genes is controlled by a cytomegalovirus promoter with an adjacent tetracycline response elements. A Tet-Off® Advanced system is used to regulate the expression of the HLA miRNA (see e.g., Product Sheet: “Tet-On® and Tet-Off® Advanced Inducible Gene Expression Systems” available from Clontech Laboratories, Inc. Mountain View, Calif., which is incorporated herein by reference).

The expression system actively transcribes the HLA miRNA in the absence of doxycycline (a stable analog of tetracycline), and ceases transcription of the HLA miRNA when doxycycline is administered. Expression of allogeneic HLA miRNA in HSC leads to inhibition of translation of the allogeneic HLA mRNA and reduces allogeneic HLA protein expression on the cell surface (see e.g., Sul et al., PNAS USA 99: 5515-5520, (2002), which is incorporated herein by reference). HSC that express HLA miRNA are tested for the presence, or lack thereof, of cell surface HLA proteins using immunohistochemistry and flow cytometry. Antibodies specific for allogeneic and haplo-identical HLA antigens (HLA-specific antibodies are available from BioLegend, San Diego, Calif.) are used to compare HLA levels on cells that are expressing HLA miRNA (no doxycycline) to cells not expressing HLA miRNA (with doxycycline).

To promote a graft versus tumor immune response the lentiviral expression vector also contains an operon that directs the expression of a therapeutic protein, interleukin-21 (IL-21) (see e.g., Parrish-Novak et al., J. Leukocyte Biol. 72: 856-863 (2002), which is incorporated herein by reference). The IL-21 gene is introduced into the lentiviral expression construct and is constitutively expressed by HSC transfected with the lentiviral vector. Transfected HSC secrete IL-21 and promote an antitumor immune response (see e.g., Hinrichs et al., Blood 111: 5326-5333, (2008), which is incorporated herein by reference).

Allogeneic HSC stably transduced with a lentiviral vector encoding allogeneic HLA miRNA, IL-21, and tetracycline regulatory elements, are infused into a patient with NHL who is receiving a non-myeloablative transplant. Methods to transplant hematopoietic stem cells into humans are known in the art (see e.g., Rizzieri et al., J. Clinical Oncology 25: 690-697, (2007), which is incorporated herein by reference). Nonmyeloablative treatment of the recipient prior to transplant on day 0 includes alemtuzumab (available from Bayer HealthCare Pharmaceuticals Inc., Wayne, N.J.), 20 mg/day infused on days −4 to 0; fludarabine (available from Bayer HealthCare Pharmaceuticals Inc., Wayne, N.J.), 30 mg/m² per day infused on days −5 to −2; cyclophosphamide (available from Baxter Healthcare Corporation Deerfield, Ill.), 500 mg/m² infused on days −5 to −2; and filgrastim (available from Amgen Inc., Thousand Oaks, Calif.), 5 mg/kg administered on day +1 until absolute neutrophil count is more than 1×10⁹/L for 2 days. Approximately 1.35×10⁷CD34⁺ HSC/kg are infused into the patient, and the patient is monitored closely for acute rejection of the grafted cells, chronic rejection, graft versus host disease, and microbial infections. Blood samples are withdrawn to assess engraftment. Short tandem repeats are analyzed using polymerase chain reaction to determine the % of donor and recipient blood cells (see e.g., the report: “Chimerism by STR Genotyping” available from ARUP Laboratories, Salt Lake City, Utah, which is incorporated herein by reference.)

To protect the patient receiving HSC from uncontrolled proliferation of the transfected HSC or other potential adverse events (e.g., GVHD), HSC expression of the allogeneic HLA miRNA may be inhibited by the administration of doxycycline to the HSC recipient. Without HLA miRNA to block allogeneic HLA translation, the protein levels are restored, and allogeneic HLA are expressed on the cell surface of the HSC, thus making the cells vulnerable to alloreactive T cells. An alloreactive cell-mediated immune response then eliminates the transfected HSC (see e.g., Game and Lechler, Transplant Immunology 10: 101-108, (2002), which is incorporated herein by reference).

### Prophetic Example 4

### MHC-Less Cells for Reducing Graft Rejection

A patient with acute myelogenous leukemia (AML) is treated by transplantation with modified peripheral blood stem cells (PBSC). Allogeneic PBSC are modified to reduce expression of MHC Class I (MHC I) proteins by expression of a viral gene that targets MHC I proteins for destruction. The PBSC cells are also engineered to express a therapeutic protein, interleukin-15 (IL-15). Peripheral blood stem cells are transduced with a lentiviral expression vector encoding cytomegalovirus (CMV) protein, unique sequence 11 (US11), to target MHC I proteins for destruction and avoid allograft rejection (see e.g., Lin et al., Cellular and Molecular Immunology 4: 91-98, (2007), which is incorporated herein by reference). The lentiviral vector also encodes IL-15 to promote donor cell killing of leukemia cells (see e.g., Zhang et al., Cytokine 42: 128-136, (2008), which is incorporated herein by reference).

The US11 and IL-15 genes are controlled by promoters with transcriptional response elements. For example, the Tet-Off® system regulates the expression of US11; and Tet-Off® directs constitutive expression of US11 until doxycycline is administered. Doxycycline binds a transcriptional transactivator protein and inhibits transcription of US11 (see e.g., Product Sheet: “Tet-On® and Tet-Off® Advanced Inducible Gene Expression Systems” available from Clontech Laboratories, Inc. Mountain View, Calif. which is incorporated herein by reference). Transcription of the IL-15 gene is controlled by a human metallothionien II (MT-II) transcriptional regulatory system; IL-15 expression requires MT-II induction by provision of zinc chloride.

Methods to obtain PBSC from peripheral blood are known in the art (see e.g., Lane et al., Blood 85: 275-282, (1995) which is incorporated herein by reference). To mobilize PBSC the donor is given granulocyte colony-stimulating factor (G-CSF; also known as filgrastim from Amgen Inc., Thousand Oaks, Calif.) 10 μg/kg/day subcutaneously for 4 days. PBSC are harvested by leukapheresis on day 5 (see e.g., Lane et al., Ibid.). PBSC are selected using magnetic beads and anti-CD34 antibodies (Magnetic beads, antibodies and protocols are available from Miltenyi Biotec, Bergisch Gladbach, Germany.). Approximately 10⁸ mononuclear CD34⁺ cells are obtained, and they are modified by transduction with a lentiviral expression vector.

A lentiviral expression vector is constructed to encode human CMV US11, a viral protein that targets MHC I heavy chains for degradation (see e.g., Lin et al., Cell. and Mol. Immunol. 4: 91-98, (2007), and Hansen et al., Science 328: 102-106, (2010), each of which is incorporated herein by reference.) For example, cell surface expression of MHC I proteins from the HLA-A, -B, and -C loci are blocked by expression of US11. The expression of US11 is controlled by tetracycline responsive regulatory elements in combination with a minimal CMV promoter. Lentiviral vectors for expression of target genes in primary cells and nondividing cells are known in the art. See e.g., User Manual: “Lenti-XTM Tet-Off® Advanced Inducible Expression System” available from Clontech Laboratories, Inc. Mountain View, Calif., which is incorporated herein by reference. DNA sequences encoding US11 (see e.g., Lin et al., Ibid.) are cloned into a plasmid-based expression vector containing required elements for packaging the expression construct into virions. The plasmid is combined with a packaging mixture and transfected into a 293T cell line (available from American Type Culture Collection, Manassas, Va.) to produce a recombinant, nonreplicating lentivirus. Lentiviral stocks with a titer of approximately 10⁵ to 10⁷ transducing units/ml are sufficient to transduce 10⁶-10⁸ PBSC at a multiplicity of infection of 1.0. To determine the titer of the lentivirus stock, serial ten-fold dilutions of the stock are applied to a HT-1080 cell line (available from American Type Culture Collection, Manassas, Va.) and the number of transduced cells is counted after growth in puromycin. Puromycin resistance gene is incorporated in the lentiviral expression vector to allow selection of stably transduced cells. Expression of US11 is controlled by a promoter with adjacent tetracycline response elements. A Tet-Off® Advanced system is used to regulate the expression of the US11 (see e.g., Product Sheet: “Tet-On® and Tet-Off® Advanced Inducible Gene Expression Systems” available from Clontech Laboratories, Inc. Mountain View, Calif., which is incorporated herein by reference). The expression system actively transcribes US11 in the absence of doxycycline (a stable analog of tetracycline) and ceases transcription of the US11 when doxycycline is administered. Expression of US11 protein in PBSC leads to degradation of MHC I proteins resulting in their disappearance from the PBSC surface (see e.g., Lin et al., Ibid. and Hansen et al., Ibid.). PBSC that express US11 are tested for the presence of cell surface HLA proteins using mixed lymphocyte cultures, immunohistochemistry, and flow cytometry. Methods to detect HLA-A, -B, and -C antigens on lymphocytes are known in the art (see e.g., U.S. Patent App. Pub. No. 2009/0285842, which is incorporated herein by reference).

Mixed lymphocyte cultures are performed with peripheral blood lymphocytes (PBL) from the recipient (the AML patient) and modified PBSC that are pre-treated with mitomycin C (MMC) to block cell proliferation. Approximately 10⁴ modified PBSC are combined with 10⁵ recipient PBL in a well of a 96 well plate with media containing RPMI 1640, HEPES (25 mmol/L), L-Glutamine (2 mmol/L), 10% fetal bovine serum and 10% antibiotics. The cells are cultured for 6 days at 37° C. with 5% CO₂ in air and then counted using a ViCell-XR™ cell counter (Beckman Coulter, Inc., Brea, Calif.). Flow cytometry to detect HLA antigens is done using a flow cytometer model XL (from Beckman Coulter, Inc., Miami, Fla.) with protocols and reagents provided by Beckman Coulter.

Antibodies specific for HLA-A, -B and -C antigens (HLA-specific antibodies are available from BioLegend, San Diego, Calif.) are used to assay for HLA antigen levels on modified PBSC cells that are expressing US11 (no doxycycline), modified PBSC cells not expressing US11 (with doxycycline), and control cells (untreated PBSC). Modified PBSC that display minimal HLA antigens on their cell surface and minimal stimulation of recipient PBL proliferation in mixed lymphocyte cultures are characterized further to assess the expression of IL-15.

The lentiviral expression vector is designed to also contain an operon that directs the expression of a therapeutic protein, IL-15. The IL-15 gene is introduced in the lentiviral expression construct under the control of the human MT-II transcriptional regulatory system. Methods for constructing human MT-II promoters and vectors and for placing genes under MT-II regulation are known in the art (see e.g., U.S. Pat. No. 4,601,978, which is incorporated herein by reference). PBSC are transduced with the lentiviral vector containing the MT-II promoter sequences fused to the IL-15 gene and the tetracycline-regulated US11 gene (see above). PBSC transduced with the MT-II IL-15 construct are induced by provision of ZnCl₂ at 10⁻⁴ M to 10⁻⁶ M. ZnCl₂ induces secretion of IL-15 protein, which promotes an antitumor immune response, particularly a NK cell response (see e.g., Zhang et al., Ibid.). AML patients who are transplanted with genetically modified PBSC are given 25 mM ZnCl₂ orally to induce IL-15 expression by the genetically modified PBSC. Interleukin-15 promotes NK cell cytolytic activity by stimulating NK cell expression of an activation receptor, NKG2D (see e.g., Zhang et al., Ibid.). NKG2D recognizes protein ligands expressed by cells undergoing genotoxic stress. For example, AML tumor cells subjected to chemotherapy express NKG2D ligands may include MICA, MICB, ULBP1, ULBP2, ULBP3, and ULBP4 (see e.g., Caligiuri et al., Blood 112: 461-469, (2008), which is incorporated herein by reference).

Allogeneic PBSC stably transduced with a lentiviral vector encoding US11, IL-15 and Fas are infused into a patient with AML who is receiving a non-myeloablative transplant. Methods to transplant hematopoietic stem cells into humans are known in the art (see e.g., Rizzieri et al., J. Clinical Oncology 25: 690-697, (2007), which is incorporated herein by reference).

Nonmyeloablative treatment of the recipient prior to transplant on day 0 includes alemtuzumab (available from Bayer HealthCare Pharmaceuticals Inc., Wayne, N.J.), 20 mg/day infused on days −4 to 0; fludarabine (available from Bayer HealthCare Pharmaceuticals Inc., Wayne, N.J.), 30 mg/m² per day infused on days −5 to −2; cyclophosphamide (available from Baxter Healthcare Corporation Deerfield, Ill.), 500 mg/m² infused on days −5 to −2; and filgrastim (available from Amgen Inc., Thousand Oaks, Calif.), 5 mg/kg administered on day +1 until the absolute neutrophil count is more than 1×10⁹/L for 2 days. Approximately 1.35×10⁷CD34⁺ PBSC/kg are infused into the patient and the patient is monitored closely for acute rejection of the grafted cells, chronic rejection, graft versus host disease, and microbial infections. Blood samples are drawn to assess engraftment. Short tandem repeats are analyzed using polymerase chain reaction to determine the percentage of donor and recipient blood cells (see e.g., the report: “Chimerism by STR Genotyping” available from ARUP Laboratories, Salt Lake City, Utah, which is incorporated herein by reference).

To safeguard the AML patient receiving modified PBSC in the event of uncontrolled proliferation of the modified PBSC, or other potential adverse events (e.g., GVHD), US11 expression is inhibited by administration of doxycycline. Modified PBSC without US11 protein to block allogeneic HLA expression will restore allogeneic HLA antigens on the cell surface, thus making the cells vulnerable to alloreactive T cells. An alloreactive cell-mediated immune response eliminates the transfected HSC (see e.g., Game and Lechler, Transplant Immunol. 10: 101-108, (2002), which is incorporated herein by reference). Alternatively, the AML patient may be given doxycycline to allow allogeneic HLA antigen expression and an antibody reactive with a donor-derived specific allogeneic HLA antigen (HLA-specific antibodies are available from BioLegend, San Diego, Calif.).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A composition, comprising:
a modified eukaryotic cell including at least one modification sufficient to reduce or eliminate expression of at least one endogenous histocompatibility antigen related gene; and at least one detection material.

2. The composition of claim 1, wherein the at least one detection material includes at least one of a photo-activatable substance, nanoparticle, radioactive substance, luminescent substance, colorimetric substance, odorous substance, x-ray absorber, ultrasound absorber, or electronic identification device.

3. The composition of claim 2, wherein the electronic identification device includes at least one radio frequency identification device (RFID).

4. The composition of claim 1, wherein the at least one detection material includes at least one of a diamagnetic particle, ferromagnetic particle, paramagnetic particle, super paramagnetic particle, particle with altered isotope, or other magnetic particle.

5. The composition of claim 1, further comprising at least one Natural Killer cell inhibitor.

6. The composition of claim 1, wherein the modified eukaryotic cell includes at least one somatic cell. In an embodiment, the modified eukaryotic cell includes at least one of a blood cell, muscle cell, nerve cell, fibroblast, adipose cell, stem cell, pluripotent cell, epithelial cell, skin cell, liver cell, spleen cell, oocyte, Sertoli cell, neoplastic cell, hematopoietic stem cell, antigen presenting cell, lymphocyte, thymocyte, neuronal stem cell, sperm cell, retinal cell, pancreatic cell, osteoclast, osteoblast, myocyte, embryonic stem cell, keratinocyte, mucosal cell, mesenchymal stem cell, T cell, B cell, memory T cell, memory B cell, lymphocyte, thymocyte, or other cell.

7. A method of administering at least one modified cell to at least one biological tissue, comprising:
providing a composition to at least one biological tissue;
wherein the composition includes a modified eukaryotic cell including at least one modification sufficient to reduce or eliminate expression of at least one endogenous histocompatibility antigen related gene; and at least one detection material.

8. The method of claim 7, wherein the origin of the biological tissue is of a different subject than the modified eukaryotic cell.

9. The method of claim 7, wherein the origin of the biological tissue is of a different species than the modified eukaryotic cell.

10.-13. (canceled)

14. A composition, comprising:
a modified eukaryotic cell including at least one regulatory nucleic acid construct including an operon with an inducible promoter and encoding a regulatory gene product that is sufficient to modulate the expression of at least one endogenous eukaryotic cell histocompatibility antigen related gene, the modified eukaryotic cell further including at least one cell death-initiating component sufficient to initiate death of the at least one modified eukaryotic cell.

15. The composition of claim 14, further comprising at least one Natural Killer cell inhibitor.

16. The composition of claim 14, wherein the at least one cell death-inducing component includes at least one cell death-initiating nucleic acid construct.

17. The composition of claim 16, wherein the at least one cell death-initiating nucleic acid construct includes at least one inducible regulatory element.

18. The composition of claim 16, wherein the at least one cell death-initiating nucleic acid construct encodes at least one gene product sufficient to initiate death of the modified cell.

19. The composition of claim 16, wherein the at least one cell death-initiating nucleic acid construct is configured to initiate programmed cell death of the cell.

20. The composition of claim 16, wherein the at least one cell death-initiating nucleic acid construct is configured to initiate at least one of necrosis, pyroptosis, autophagocytosis, or apoptosis of the cell.

21. The composition of claim 16, wherein the at least one cell death-initiating nucleic acid construct encodes at least one programmed cell death gene product.

22. The composition of claim 16, wherein the at least one cell death-initiating nucleic acid construct encodes at least one of programmed cell death 1 gene (PDCD1), programmed cell death 2 gene (PDCD2), programmed cell death 3 gene (PDCD3), programmed cell death 4 gene (PDCD4), programmed cell death 5 gene (PDCD5), programmed cell death 6 gene (PDCD6), programmed cell death 7 gene (PDCD7), programmed cell death 8 gene (PDCD8), programmed cell death 9 gene (PDCD9), programmed cell death 10 gene (PDCD10), programmed cell death 11 gene (PDCD11), programmed cell death 12 gene (PDCD12), caspase gene, rel gene, hok gene, sok gene, diaminopimelate gene, nuclease gene, methylase gene, DNA ligase gene, DNA gyrase gene, toxin-antitoxin module, relF gene, triclosan, lysine, lysine-holin, Bcl-2-associated X protein (Bax), Bcl-2-associated death promoter (BAD), Bcl-2-homologous antagonist/killer (Bak), Bcl-2-related ovarian killer protein (Bok), Fas ligand, Fas receptor, or a foreign histocompatibility gene.

23. The composition of claim 22, wherein the toxin-antitoxin module includes at least one of masEF, chpBIK, relBE, yefM-yoeB, dinJ-yaf1, or ecnA-ecnB.

24. The composition of claim 16, wherein the at least one cell death-initiating nucleic acid construct includes at least one gene product configured to lyse the at least one cell.

25. The composition of claim 24, wherein the at least one gene product to lyse the at least one cell includes at least one of a nuclease gene, or lysis gene E.

26. The composition of claim 16, wherein the at least one cell death-initiating nucleic acid construct encodes at least one gene product configured to interfere with the utilization of at least one cellular metabolite.

27. The composition of claim 14, wherein the at least one cell death-initiating component includes at least one receptor configured to allow entry of at least one of a toxin or pathogen.

28. The composition of claim 14, wherein the at least one cell death-initiating component includes at least one energy absorbing structure.

29. The composition of claim 28, wherein the energy absorbing structure includes at least one of an x-ray absorber, metal nanoparticle, or ultrasound absorber.

30. A method of administering at least one modified cell to at least one biological tissue, comprising:
providing a composition to at least one biological tissue;
wherein the composition includes a modified eukaryotic cell including at least one regulatory nucleic acid construct including an operon with an inducible promoter and encoding a regulatory gene product that is sufficient to Modulate the expression of at least one endogenous eukaryotic cell histocompatibility antigen related gene, the modified eukaryotic cell further including at least one cell death-initiating component sufficient to initiate death of the at least one modified eukaryotic cell.

31. The method of claim 30, wherein the origin of the biological tissue is of a different subject than the modified eukaryotic cell.

32. The method of claim 30, wherein the origin of the biological tissue is of a different species than the modified eukaryotic cell.

33.-48. (canceled)

49. A composition, comprising:
a modified eukaryotic cell including at least one regulatory nucleic acid construct including an operon with an inducible promoter and encoding a regulatory gene product that is sufficient to modulate the expression of at least one endogenous eukaryotic cell histocompatibility antigen related gene, and including at least one cell death-inducing component.

50. The composition of claim 49, wherein the at least one cell death-inducing component includes at least one cell death-initiating nucleic acid construct.

51. The composition of claim 50, wherein the at least one cell death-initiating component includes at least one receptor configured to allow entry of at least one of a toxin or pathogen.

52. The composition of claim 50, wherein the at least one cell death-initiating component includes at least one energy absorbing structure.

53. The composition of claim 50, wherein the energy absorbing structure includes at least one of an x-ray absorber, metal nanoparticle, or ultrasound absorber.

54. A method of administering at least one modified cell to at least one biological tissue, comprising:
providing a composition to at least one biological tissue;
wherein the composition includes a modified eukaryotic cell including at least one regulatory nucleic acid construct including an operon with an inducible promoter and encoding a regulatory gene product that is sufficient to modulate the expression of at least one endogenous eukaryotic cell histocompatibility antigen related gene, and including at least one cell death-inducing component.

55. The method of claim 54, wherein the origin of the biological tissue is of a different subject than the modified eukaryotic cell.

56. The method of claim 54, wherein the origin of the biological tissue is of a different species than the modified eukaryotic cell.

57.-60. (canceled)
